SEQUENCE LISTING (2 sequences)

SEQ ID NO: 1 - protein, 2356 aa, Homo sapiens
MAHHHHHHDEVDDEPSPLAQPLELNQHSRFIIGSVSEDNSEDEISNLVKLDLLEKEGSLSPASVGSDTLSDLGISSLQDGLALHIRSSMSGLHLVKQGRDRKKIDSQRDFTVASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGVVCGALHVADVSLRNSVSNFLHSLERGQVLPAHTLLNTVDVELIYEGVKYVLKVTRQSPNSYVVIMNGSCVEVDVHRLSDGGLLLSYDGSSYTTYMKEEVDRYRITIGNKTCVFEKENDPSVMRSPSAGKLIQYIVEDGGHVFAGQCYAEIEVMKMVMTLTAVESGCIHYVKRPGAALDPGCVLAKMQLDNPSKVQQAELHTGSLPRIQSTALRGEKLHRVFHYVLDNLVNVMNGYCLPDPFFSSKVKDWVERLMKTLRDPSLPLLELQDIMTSVSGRIPPNVEKSIKKEMAQYASNITSVLCQFPSQQIANILDSHAATLNRKSEREVFFMNTQSIVQLVQRYRSGIRGHMKAVVMDLLRQYLRVETQFQNGHYDKCVFALREENKSDMNTVLNYIFSHAQVTKKNLLVTMLIDQLCGRDPTLTDELLNILTELTQLSKTTNAKVALRARQVLIASHLPSYELRHNQVESIFLSAIDMYGHQFCIENLQKLILSETSIFDVLPNFFYHSNQVVRMAALEVYVRRAYIAYELNSVQHRQLKDNTCVVEFQFMLPTSHPNRGNIPTLNRMSFSSNLNHYGMTHVASVSDVLLDNSFTPPCQRMGGMVSFRTFEDFVRIFDEVMGCFSDSPPQSPTFPEAGHTSLYDEDKVPRDEPIHILNVAIKTDCDIEDDRLAAMFREFTQQNKATLVDHGIRRLTFLVAQKDFRKQVNYEVDRRFHREFPKFFTFRARDKFEEDRIYRHLEPALAFQLELNRMRNFDLTAIPCANHKMHLYLGAAKVEVGTEVTDYRFFVRAIIRHSDLVTKEASFEYLQNEGERLLLEAMDELEVAFNNTNVRTDCNHIFLNFVPTVIMDPSKIEESVRSMVMRYGSRLWKLRVLQAELKINIRLTPTGKAIPIRLFLTNESGYYLDISLYKEVTDSRTAQIMFQAYGDKQGPLHGMLINTPYVTKDLLQSKRFQAQSLGTTYIYDIPEMFRQSLIKLWESMSTQAFLPSPPLPSDMLTYTELVLDDQGQLVHMNRLPGGNEIGMVAWKMTFKSPEYPEGRDIIVIGNDITYRIGSFGPQEDLLFLRASELARAEGIPRIYVSANSGARIGLAEEIRHMFHVAWVDPEDPYKGYRYLYLTPQDYKRVSALNSVHCEHVEDEGESRYKITDIIGKEEGIGPENLRGSGMIAGESSLAYNEIITISLVTCRAIGIGAYLVRLGQRTIQVENSHLILTGAGALNKVLGREVYTSNNQLGGIQIMHNNGVTHCTVCDDFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSIPADPANLDSEAKIIQQAGQVWFPDSAFKTYQAIKDFNREGLPLMVFANWRGFSGGMKDMYDQVLKFGAYIVDGLRECCQPVLVYIPPQAELRGGSWVVIDSSINPRHMEMYADRESRGSVLEPEGTVEIKFRRKDLVKTMRRVDPVYIHLAERLGTPELSTAERKELENKLKEREEFLIPIYHQVAVQFADLHDTPGRMQEKGVISDILDWKTSRTFFYWRLRRLLLEDLVKKKIHNANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILSTMDSPST

SEQ ID NO: 2 - protein, 2458 aa, Homo sapiens
MVLLLCLSCLIFSCLTFSWLKIWGKMTDSKPITKSKSEANLIPSQEPFPASDNSGETPQRNGEGHTLPKTPSQAEPASHKGPKDAGRRRNSLPPSHQKPPRNPLSSSDAAPSPELQANGTGTQGLEATDTNGLSSSARPQGQQAGSPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGVVCGALNVADAMFRTCMTDFLHSLERGQVLPADSLLNLVDVELIYGGVKYILKVARQSLTMFVLIMNGCHIEIDAHRLNDGGLLLSYNGNSYTTYMKEEVDSYRITIGNKTCVFEKENDPTVLRSPSAGKLTQYTVEDGGHVEAGSSYAEMEVMKMIMTLNVQERGRVKYIKRPGAVLEAGCVVARLELDDPSKVHPAEPFTGELPAQQTLPILGEKLHQVFHSVLENLTNVMSGFCLPEPVFSIKLKEWVQKLMMTLRHPSLPLLELQEIMTSVAGRIPAPVEKSVRRVMAQYASNITSVLCQFPSQQIATILDCHAATLQRKADREVFFINTQSIVQLVQRYRSGIRGYMKTVVLDLLRRYLRVEHHFQQAHYDKCVINLREQFKPDMSQVLDCIFSHAQVAKKNQLVIMLIDELCGPDPSLSDELISILNELTQLSKSEHCKVALRARQILIASHLPSYELRHNQVESIFLSAIDMYGHQFCPENLKKLILSETTIFDVLPTFFYHANKVVCMASLEVYVRRGYIAYELNSLQHRQLPDGTCVVEFQFMLPSSHPNRMTVPISITNPDLLRHSTELFMDSGFSPLCQRMGAMVAFRRFEDFTRNFDEVISCFANVPKDTPLFSEARTSLYSEDDCKSLREEPIHILNVSIQCADHLEDEALVPILRTFVQSKKNILVDYGLRRITFLIAQEKEFPKFFTFRARDEFAEDRIYRHLEPALAFQLELNRMRNFDLTAVPCANHKMHLYLGAAKVKEGVEVTDHRFFIRAIIRHSDLITKEASFEYLQNEGERLLLEAMDELEVAFNNTSVRTDCNHIFLNFVPTVIMDPFKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAIKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

(19) United States
(12) Patent Application Publication
(10) Pub. No.: US 2012/0225900 A1
(43) Pub. Date: Sep. 6, 2012

(54) N2-PYRAZOLOSPIROKETONE ACETYL-COA CARBOXYLASE INHIBITORS

(75) Inventors: Scott William Bagley, Mystic, CT (US); David Andrew Griffith, Old Saybrook, CT (US); Daniel Wei-Shung Kung, Salem, CT (US)

(73) Assignee: PFIZER INC., New York, NY (US)

(21) Appl. No.: 13/508,744

(22) PCT Filed: Oct. 28, 2010
(86) PCT No.: PCT/IB2010/054893

§ 371 Date: May 9, 2012

Related U.S. Application Data
(60) Provisional application No. 61/259,831, filed on Nov. 10, 2009.

(51) Int. Cl. A61K 31/438; A61P 3/04; A61P 3/06; C07D 471/10; A61P 3/10

(52) U.S. Cl. 514/278; 546/17

(57) ABSTRACT

The invention provides a compound of Formula (I) or a pharmaceutically acceptable salt of said compound, wherein R¹, R², R³ and R⁴ are as described herein; pharmaceutical compositions thereof; and the use thereof in treating diseases, conditions or disorders modulated by the inhibition of an acetyl-CoA carboxylase enzyme(s) in an animal.

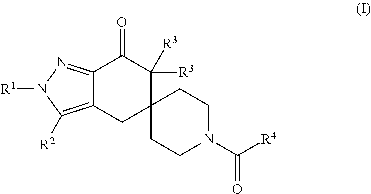

### FIELD OF THE INVENTION

This invention relates to substituted pyrazolospiroketone compounds that act as an inhibitor of acetyl-CoA carboxylases and their use in treating diseases, conditions or disorders modulated by the inhibition of acetyl-CoA carboxylase enzyme(s).

### BACKGROUND OF THE INVENTION

Acetyl-CoA carboxylases (ACC) are a family of enzymes found in most species and are associated with fatty acid synthesis and metabolism through catalyzing the production of malonyl-CoA from acetyl-CoA. In mammals, two isoforms of the ACC enzyme have been identified. ACC1, which is expressed at high levels in lipogenic tissues, such as fat and the liver, controls the first committed step in the biosynthesis of long-chain fatty acids. If acetyl-CoA is not carboxylated to form malonyl-CoA, it is metabolized through the Krebs cycle. ACC2, which is a minor component of hepatic ACC but the predominant isoform in heart and skeletal muscle, and catalyzes the production of malonyl-CoA at the cytosolic surface of mitochondria, and regulates how much fatty acid is utilized in β-oxidation by inhibiting carnitine palmitoyl transferase. Thus, by increasing fatty acid utilization and by preventing increases in de novo fatty acid synthesis, chronic administration of an ACC inhibitor (ACC-I) may also deplete liver and adipose tissue triglyceride (TG) stores in obese subjects consuming a high or low-fat diet, leading to selective loss of body fat.

Studies conducted by Abu-Etheiga, et al., suggest that ACC2 plays an essential role in controlling fatty acid oxidation and, as such it would provide a target in therapy against obesity and obesity-related diseases, such as type-2 diabetes. See, Abu-Etheiga, L., et al., “Acetyl-CoA carboxylase 2 mutant mice are protected against obesity and diabetes induced by high-fat/high-carbohydrate diets” PNAS, 100(18) 10207-10212 (2003). See also, Choi, C. S., et al., “Continuous fat oxidation in acetyl-CoA carboxylase 2 knockout mice increases total energy expenditure, reduces fat mass, and improves insulin sensitivity” PNAS, 104(42) 16480-16485 (2007).

It is becoming increasingly clear that hepatic lipid accumulation causes hepatic insulin resistance and contributes to the pathogenesis of type 2 diabetes. Salvage, et al., demonstrated that ACC1 and ACC2 are both involved in regulating fat oxidation in hepatocytes while ACC1, the dominant isoform in rat liver, is the sole regulator of fatty acid synthesis. Furthermore, in their model, combined reduction of both isoforms is required to significantly lower hepatic malonyl-CoA levels, increase fat oxidation in the fed state, reduce lipid accumulation, and improve insulin action in vivo. Thus, showing that hepatic ACC1 and ACC2 inhibitors may be useful in the treatment of nonalcoholic fatty liver disease (NAFLD) and hepatic insulin resistance. See, Savage, D. B., et al., “Reversal of diet-induced hepatic steatosis and hepatic insulin resistance by antisense oligonucleotide inhibitors of acetyl-CoA carboxylases 1 and 2” J Clin Invest doi: 10.1172/JCI27300. See also, Oh, W., et al., “Glucose and fat metabolism in adipose tissue of acetyl-CoA carboxylase 2 knockout mice” PNAS, 102(5) 1384-1389 (2005).

Consequently, there is a need for medicaments containing ACC1 and/or ACC2 inhibitors to treat obesity and obesity-related diseases (such as, NAFLD and type-2 diabetes) by inhibiting fatty acid synthesis and by increasing fatty acid oxidation.

### SUMMARY OF THE INVENTION

The present invention relates to compounds having the structure of Formula (I)

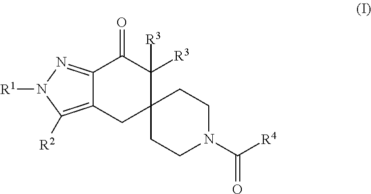

wherein R¹ is (C₁-C₆)alkyl, (C₃-C₇)cycloalkyl, tetrahydrofuranyl or oxetanyl; wherein said (C₁-C₆)alkyl is optionally substituted with 1 to 2 substituents independently selected from (C₁-C₃)alkoxy; hydroxy, halo, phenyl, tetrahydrofuranyl or oxetanyl;

R² is hydrogen, halo, (C₁-C₃)alkyl, cyano or —C(═NH)(OCH₃);

R³ are each independently hydrogen or (C₁-C₃)alkyl;

R⁴ is (C₆-C₁₀)aryl, 5 to 12 membered heteroaryl or 8 to 12 membered fused heterocyclicaryl; wherein said (C₆-C₁₀)aryl, 5 to 12 membered heteroaryl or 8 to 12 membered fused heterocyclicaryl are each optionally substituted with one to three substituents independently selected from (C₁-C₃)alkyl, (C₁-C₃)alkoxy, halo, amino, (C₁-C₃)alkylamino, di(C₁-C₃)alkylamino, hydroxy, cyano, amido, phenyl, 5 to 6 membered heteroaryl or 5 to 6 membered heterocyclyl; or a pharmaceutically acceptable salt thereof. A preferred embodiment of the present invention are compounds of Formula (I) wherein R⁴ is (C₆-C₁₀) aryl selected from phenyl or naphthyl; a 5 to 12 membered heteroaryl selected from pyridinyl, pyrazolyl, pyrimidinyl, triazolyl, indolizinyl, indazolyl, pyrrolo[2,3-b]pyridinyl, pyrrolo[3,2-b]pyridinyl, pyrrolo[1,2-a]pyrazinyl, imidazo[1,2-a]pyridinyl, imidazo[1,5-a]pyridinyl, benzo[d]imidazolyl, pyrazolo[3,4-b]pyridinyl, pyrazolo[4,3-b]pyridinyl, pyrazolo[1,5-a]pyrimidinyl, benzo[d]imidazol-2-onyl, 1,6-naphthyridinyl, quinoxalinyl, quinolin-4-onyl or isoquinolin-1-onyl; or an 8 to 12 membered fused heterocyclicaryl selected from 3,4-dihydroquinolin-2-onyl or indolin-2-onyl; wherein each R⁴ group is optionally substituted with one to four substituents independently selected from (C₁-C₃)alkyl, (C₁-C₃)alkoxy, halo, amino, (C₁-C₃)alkylamino, di(C₁-C₃)alkylamino, hydroxy, cyano, amido, phenyl, 5 to 6 membered heteroaryl or 5 to 6 membered heterocyclyl; or a pharmaceutically acceptable salt thereof.

Another preferred embodiment of the present invention is the compound of Formula (I) wherein R¹ is (C₁-C₆)alkyl, (C₃-C₇)cycloalkyl, or tetrahydrofuranyl; and R² is hydrogen or methyl; or a pharmaceutically acceptable salt thereof. Yet another preferred embodiment of the present invention is the compound of Formula (I) wherein R¹ is ethyl, isopropyl or t-butyl; and R⁴ is phenyl, pyrazolyl, imidazolyl, triazolyl, pyridinyl, pyrimidinyl, indolyl, benzopyrazinyl, benzoimidazolyl, benzoimidazolonyl, pyrrolopyridinyl, pyrrolopyrimidinyl, pyrazolopyridinyl, pyrazolopyrimidinyl, indazolyl, indolinonyl, naphthyridinyl, quinolinyl, quinolinonyl, dihydroquinolinonyl, oxo-dihydroquinolinonyl, isoquinolinyl, isoquinolinonyl, dihydroisoquinonyl or oxo-dihydroisoquinonyl, each optionally substituted with one to three substituents independently selected from fluoro, chloro, methyl, amino, methylamino, dimethylamino, amido, cyano, phenyl, imidazolyl, pyrazolyl, triazolyl, pyridinyl or morpholinyl; or a pharmaceutically acceptable salt thereof. A further preferred embodiment of the present invention is the compound of Formula (I) wherein R¹ is isopropyl or t-butyl; R² is hydrogen; and each R³ is hydrogen; or a pharmaceutically acceptable salt thereof. Yet another preferred embodiment of the present invention is the compound of formula (I) wherein R⁴ is indazolyl, benzoimidazolyl, 1-oxo-1,2-dihydroisoquinolinyl, 1H-pyrrolo[3,2-b]pyridinyl, 2-oxo-2,3-dihydro-1H-benzo[d]imidazolyl, 1H-pyrazolylphenyl, 1H-pyrazolylpyridinyl, or 1H-imidazolylphenyl; each optionally substituted with one to two methyl, chloro or fluoro; or a pharmaceutically acceptable salt thereof.

Another preferred embodiment of the present invention is a compound selected from 2-tert-butyl-1′-(1H-indazole-5-carbonyl)-4,6-dihydrospiro[indazole-5,4′-piperidin]-7(2H)-one; 2-tert-butyl-1′-(4-chloro-3-methyl-phenylcarbonyl)-4,6-dihydrospiro[indazole-5,4′-piperidin]-7(2H)-one; 2-tert-butyl-1′-(1H-indazole-5-carbonyl)-6,6-dimethyl-4,6-dihydrospiro[indazole-5,4′-piperidin]-7(2H)-one; 2-tert-butyl-1′-(1H-indazole-5-carbonyl)-6-methyl-4,6-dihydrospiro[indazole-5,4′-piperidin]-7(2H)-one; (R)-2-tert-butyl-1′-(1H-indazole-5-carbonyl)-6-methyl-4,6-dihydrospiro[indazole-5,4′-piperidin]-7(2H)-one; and (S)-2-tert-butyl-1′-(1H-indazole-5-carbonyl)-6-methyl-4,6-dihydrospiro[indazole-5,4′-piperidin]-7(2H)-one; or a pharmaceutically acceptable salt thereof.

Another aspect of the present invention is a pharmaceutical composition comprising an amount of a compound of Formula (I) as described in any of the embodiments; or a pharmaceutically acceptable salt thereof and a pharmaceutically acceptable excipient, diluent, or carrier. Preferably, the composition comprises a therapeutically effective amount of a compound of the present invention. The composition may also contain at least one additional pharmaceutical agent. Preferred agents include anti-diabetic agents and/or anti-obesity agents.

In yet another aspect of the present invention is a method for treating a disease, condition, or disorder mediated by the inhibition of acetyl-CoA carboxylase enzyme(s) in a mammal that includes the step of administering to a mammal, preferably a human, in need of such treatment a therapeutically effective amount of a compound of the present invention, or a pharmaceutically acceptable salt thereof or a pharmaceutical composition thereof.

Diseases, disorders, or conditions mediated by inhibitors of acetyl-CoA carboxylases include Type II diabetes and diabetes-related diseases, such as nonalcoholic fatty liver disease (NAFLD), hepatic insulin resistance, hyperglycemia, metabolic syndrome, impaired glucose tolerance, diabetic neuropathy, diabetic nephropathy, diabetic retinopathy, obesity, dyslipidemia, hypertension, hyperinsulinemia, and insulin resistance syndrome. Preferred diseases, disorders, or conditions include Type II diabetes, nonalcoholic fatty liver disease (NAFLD), hepatic insulin resistance, hyperglycemia, impaired glucose tolerance, obesity, and insulin resistance syndrome. More preferred are Type II diabetes, nonalcoholic fatty liver disease (NAFLD), hepatic insulin resistance, hyperglycemia, and obesity. Most preferred is Type II diabetes.

A preferred embodiment is a method for treating (e.g. delaying the progression or onset of) Type 2 diabetes and diabetes-related disorders in animals comprising the step of administering to an animal in need of such treatment a therapeutically effective amount of a compound of the present invention or a pharmaceutically acceptable salt thereof or a composition thereof.

Another preferred embodiment is a method for treating obesity and obesity-related disorders in animals comprising the step of administering to an animal in need of such treatment a therapeutically effective amount of a compound of the present invention or a pharmaceutically acceptable salt thereof or a composition thereof.

Yet another preferred embodiment is a method for treating nonalcoholic fatty liver disease (NAFLD) or hepatic insulin resistance in animals comprising the step of administering to an animal in need of such treatment a therapeutically effective amount of a compound of the present invention or a pharmaceutically acceptable salt thereof or a composition thereof.

Compounds of the present invention may be administered in combination with other pharmaceutical agents (in particular, anti-obesity and anti-diabetic agents described herein below). The combination therapy may be administered as (a) a single pharmaceutical composition which comprises a compound of the present invention, at least one additional pharmaceutical agent described herein and a pharmaceutically acceptable excipient, diluent, or carrier; or (b) two separate pharmaceutical compositions comprising (i) a first composition comprising a compound of the present invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

### DETAILED DESCRIPTION OF THE INVENTION

### Definitions

The phrase “therapeutically effective amount” means an amount of a compound of the present invention or a pharmaceutically acceptable salt thereof that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated” or “modulating”, or “modulate(s)”, as used herein, unless otherwise indicated, refers to the inhibition of the Acetyl-CoA carboxylases (ACC) enzyme(s) with compounds of the present invention.

The terms “mediated” or “mediating” or “mediate(s)”, as used herein, unless otherwise indicated, refers to the (i) treatment or prevention the particular disease, condition, or disorder, (ii) attenuation, amelioration, or elimination of one or more symptoms of the particular disease, condition, or disorder, or (iii) prevention or delay of the onset of one or more symptoms of the particular disease, condition, or disorder described herein, by inhibiting the Acetyl-CoA carboxylases (ACC) enzyme(s).

The term “compounds of the present invention” (unless specifically identified otherwise) refer to compounds of Formula (I) and any pharmaceutically acceptable salts of the compounds, as well as, all stereoisomers (including diastereoisomers and enantiomers), tautomers, conformational isomers, and isotopically labeled compounds. Hydrates and solvates of the compounds of the present invention are considered compositions of the present invention, wherein the compound is in association with water or solvent, respectively.

The terms “(C₁-C₆)alkyl” and “(C₁-C₃)alkyl” are alkyl groups of the specified number of carbons, from one to six or one to three carbons, respectively, which can be either straight chain or branched. For example, the term “(C₁-C₃)alkyl” has from one to three carbons and consists of methyl, ethyl, n-propyl and isopropyl.

The term “(C₃-C₇)cycloalkyl” means a cycloalkyl group with three to seven carbon atoms and consists of cyclopropyl, cyclobutyl, cyclopentyl, cyclohexyl and cycloheptyl. The term “halo” means fluoro, chloro, bromo or iodo. The term “(C₆-C₁₀o)aryl” means an aromatic group consisting of six to ten carbon atoms such as phenyl or naphthyl.

The term “5 to 12 membered heteroaryl” means a five to twelve membered aromatic group which contains at least one heteroatom selected from nitrogen, oxygen and sulfur. As used herein the point of attachment of the “5 to 12 membered heteroaryl” group is on a carbon atom of that group. The “5 to 12 membered heteroaryl” group can be either monocyclic or bicyclic. Preferred embodiments of monocyclic heteroaryls include, but are not limited to, pyrazolyl, imidazolyl, triazolyl, pyridinyl, and pyrimidinyl. Preferred embodiments of bicyclic heteroaryls include, but are not limited to, radicals of the following ring systems:

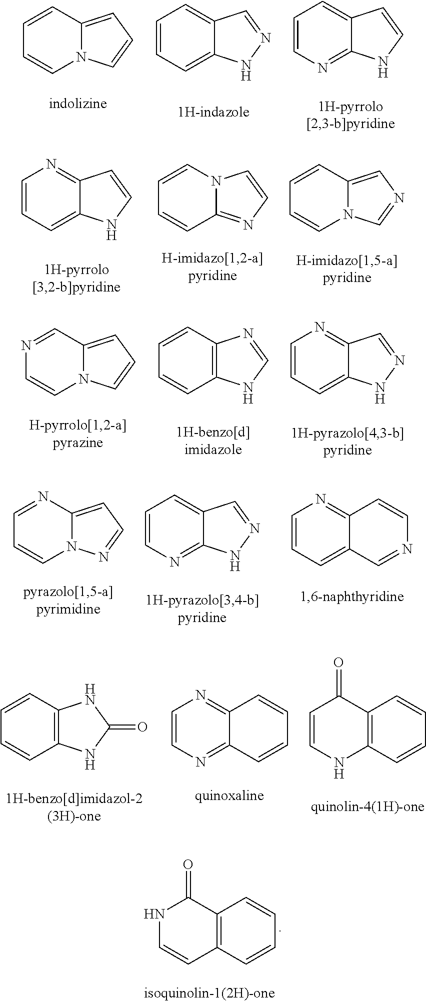

The term “8 to 12 membered fused heterocyclicaryl” means an 8 to 12 membered ring system in which a non-aromatic heterocyclic ring is fused to an aryl ring. As used herein the point of attachment of the “8 to 12 membered fused heterocyclicaryl” group is on a carbon atom of that group. A preferred embodiment includes radicals of ring systems such as:

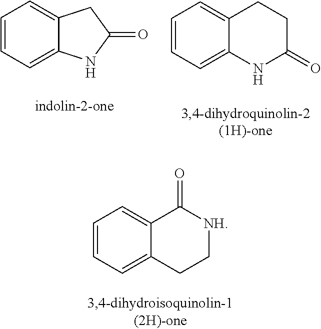

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art. For a general description of protecting groups and their use, see T. W. Greene, Protective Groups in Organic Synthesis, John Wiley & Sons, New York, 1991.

The following reaction schemes, Reaction Scheme I through Reaction Scheme II provide representative procedures that are used to prepare the compounds of Formula (I). It is to be understood that these reaction schemes are to be construed in a non-limiting manner and that reasonable variations of the depicted methods can be used to prepare the compounds of Formula (I).

Reaction Scheme I outlines the general procedures one could use to provide compounds of the present invention having Formula (Ia) which are compounds of Formula (I) in which R² and each R³ are each hydrogen. The protected spiropiperidine derivative (VIIIa) may be formed by treating the appropriately protected piperidine aldehyde (Xa) with methyl vinyl ketone (IXa). The group Pg represents an appropriate amine protecting group and is preferably N-tert-butoxycarbonyl (BOC) or carbobenzyloxy (Cbz), preferably Cbz. This reaction can be carried out in the presence of ethanolic potassium hydroxide according to a procedure analogous to that described by Roy, S. et al., Chem. Eur. J. 2006, 12, 3777-3788 at 3786. Alternatively, the reaction can be carried out in the presence of para-toluenesulfonic acid (pTSA) in refluxing benzene to provide the desired product (VIIIa).

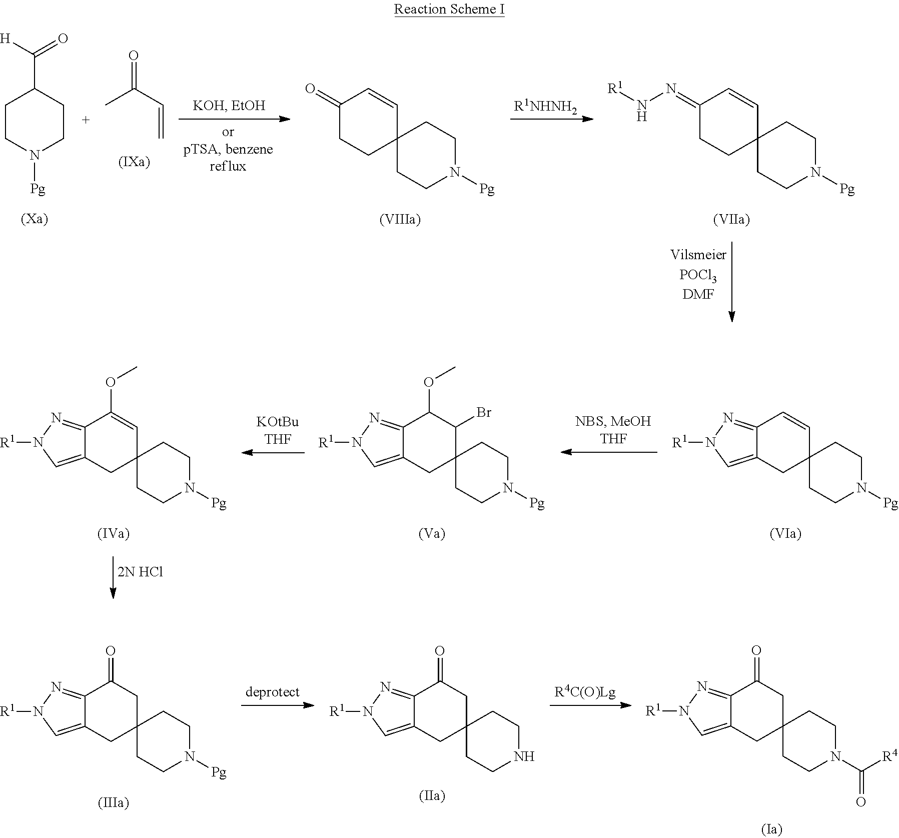

The spiropiperidine derivative (VIIIa) can then be reacted with an appropriate hydrazine derivative, R¹NHNH₂, in an appropriate solvent such as ethanol, preferably at an elevated temperature such as 60° C. to reflux to provide the functionalized spiropiperidine derivative (VIIa).

Compound (VIIa) is then reacted with a Vilsmeier reagent generated from N,N-dimethylformamide and phosphorus oxychloride in N,N-dimethylformamide, preferably at an elevated temperature such as 80° C. to provide the desired cyclized compound of formula (VIa). The compound of Formula (VIa) can then be treated with N-bromosuccinimide (NBS) in the presence of methanol in THF to provide the corresponding bromo methoxy derivative of Formula (Va). The bromo methoxy derivative (Va) is then subjected to elimination conditions using a strong base such as potassium tert-butoxide in THF to provide the compound (IVa) which is then reacted with astrong acid such as 2N HCl to provide the compound of Formula (IIIa).

The compound of Formula (IIIa) can then be deprotected to provide the free spiropiperidine derivative of Formula (IIa) using standard methods which depend on which protecting group Pg has been employed. For example, when Pg represents tert-butyloxycarbonyl (BOC) standard strong acid deprotection conditions such as 4N hydrochloric acid in dioxane or trifluoroacetic acid in an appropriate solvent such as dichloromethane can be used to remove the BOO group. When Pg represents carbobenzyloxy (Cbz), hydrogenation over palladium on carbon in ethanol or treatment with a hydrogen source such as ammonium formate or 1-methyl-1,4-cyclohexadiene in the presence of palladium on carbon in ethanol or ethyl acetate can be employed to carry out the deprotection.

The spiropiperidine derivative of Formula (IIa) can then be acylated by employing standard methods to provide the compound of Formula (Ia). For example, the compound (Ia) may then be formed using a standard peptide coupling reaction with the desired carboxylic acid (R⁴CO₂H). For example, the spiropiperidine intermediate (IIa) and carboxylic acid (R⁴CO₂H) may be coupled by forming an activated carboxylic acid ester, such as by contacting the carboxylic acid (R⁴CO₂H) with a peptide coupling reagent, such as O-(7-azabenzotriazol-1-yl)-N,N,N′,N′-tetramethyluronium hexafluorophosphate (HATU) or 1-ethyl-3-(3-dimethyllaminopropyl)carbodiimide hydrochloride (EDC.HCl), in the presence or absence of an activating agent, such as hydroxybenzotriazole (HOBt) and in the presence of a suitable base, such as N,N-diisopropylethylamine (DIEA), triethylamine or N-methylmorpholine (NMM), in a suitable solvent such as THF and/or DMF, dimethylacetamide (DMA) or dichloromethane and then contacting the activated carboxylic acid ester with the spiropiperidine derivative (IIa) to form a compound of Formula (Ia).

Alternatively, compounds of Formula (Ia) can be formed by first converting the carboxylic acid (R⁴CO₂H) to an acid chloride (R⁴COCl), such as by reacting with thionyl chloride, and then reacting the acid chloride with the spiropiperidine derivative (IIa) in the presence of an appropriate base such as triethylamine in an appropriate solvent such as dichloromethane to form a compound of Formula (Ia). Still another alternative method entails treating the carboxylic acid (R⁴CO₂H) with 2-chloro-4,6-dimethoxytriazine in the presence of a suitable base, such as N-methylmorpholine in a suitable solvent such as THF and/or DMF. To the activated ester is added a solution of the spiropiperidine derivative (IIa) and base, such as N-methylmorpholine, in a suitable solvent, such as THF and/or DMF which then provides the compound of Formula (Ia).

Reaction Scheme II provides a synthesis of compounds of Formula (Ib) starting from the intermediate of Formula (IIIb). The transformation in Reaction

Scheme IV depicts introduction of an appropriate group at the R³ position of the compound (IIIb). The compound (IIIb) is deprotonated with a strong base, such as lithium hexamethyldisilazide (LHMDS) under appropriate anhydrous conditions in an appropriate solvent, preferably at low temperature. The enolate thus formed is then reacted with an appropriate electrophile R³Lg wherein Lg represents an appropriate leaving group (such as a halide when R³Lg is an alkyl halide such as methyl iodide) to provide (IIIc) wherein R³ is an appropriate group such as an alkyl group. The deprotonation of (IIIc) and reaction with another R³Lg can then be carried out again if desired. The compound of Formula (IIIc) can then be deprotected and acylated as previously described in Reaction Scheme I to provide the compound of Formula (Ib).

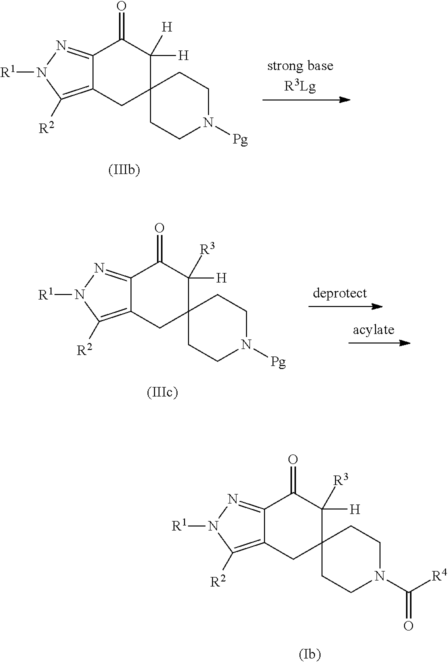

The compounds of the present invention may be isolated and used per se or in the form of their pharmaceutically acceptable salts. In accordance with the present invention, compounds with multiple basic nitrogen atoms can form salts with varying number of equivalents (“eq.”) of acid. It will be understood by practitioners that all such salts are within the scope of the present invention.

Pharmaceutically acceptable salts, as used herein in relation to compounds of the present invention, include pharmaceutically acceptable inorganic and organic salts of the compound. These salts can be prepared in situ during the final isolation and purification of a compound, or by separately reacting the compound thereof, with a suitable organic or inorganic acid and isolating the salt thus formed. Representative salts include, but are not limited to, the hydrobromide, hydrochloride, hydroiodide, sulfate, bisulfate, nitrate, acetate, trifluoroacetate, oxalate, besylate, palmitate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate and laurylsulphonate salts, and the like. These may also include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylammonium, dimethylammonium, trimethylammonium, triethylammonium, ethylammonium, and the like. For additional examples see, for example, Berge, et al., J. Pharm. Sci., 66, 1-19 (1977).

Compounds of the present invention may exist in more than one crystal form. Polymorphs of compounds of Formula (I) and salts thereof (including solvates and hydrates) form part of this invention and may be prepared by crystallization of a compound of the present invention under different conditions. For example, using different solvents or different solvent mixtures for recrystallization; crystallization at different temperatures; various modes of cooling, ranging from very fast to very slow cooling during crystallization. Polymorphs may also be obtained by heating or melting a compound of the present invention followed by gradual or fast cooling. The presence of polymorphs may be determined by solid probe nuclear magnetic resonance (NMR) spectroscopy, infrared (IR) spectroscopy, differential scanning calorimetry, powder X-ray diffraction or such other techniques.

This invention also includes isotopically-labeled compounds, which are identical to those described by Formula (1), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, sulfur and fluorine, such as ²H, ³H, ¹³C, ¹⁴C, ¹⁵N, ¹⁸O, ¹⁷O, ³⁵S, ³⁶Cl, ¹²⁵I, ¹²⁹I, and ¹⁸F respectively. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of the present invention can generally be prepared by carrying out the procedures disclosed in the schemes and/or in the Examples below, by substituting a readily available isotopically labeled reagent for a non-isotopically labeled reagent.

The compounds of the present invention may contain stereogenic centers. These compounds may exist as mixtures of enantiomers or as pure enantiomers. Wherein a compound includes a stereogenic center, the compounds may be resolved into the pure enantiomers by methods known to those skilled in the art, for example by formation of diastereoisomeric salts which may be separated, for example, by crystallization; formation of stereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, the specific stereoisomers may be synthesized by using an optically active starting material, by asymmetric synthesis using optically active reagents, substrates, catalysts or solvents, or by converting one stereoisomer into the other by asymmetric transformation.

Compounds of the present invention may exist in different stable conformational forms which may be separable. Torsional asymmetry due to restricted rotation about an asymmetric single bond, for example because of steric hindrance or ring strain, may permit separation of different conformers. The compounds of the present invention further include each conformational isomer of compounds of Formula (I) and mixtures thereof.

Compounds of the present invention are useful for treating diseases, conditions and/or disorders modulated by the inhibition of the acetyl-CoA carboxylases enzyme(s) (in particular, ACC1 and ACC2). Another embodiment of the present invention is a pharmaceutical composition comprising a therapeutically effective amount of a compound of the present invention and a pharmaceutically acceptable excipient, diluent or carrier. The compounds of the present invention (including the compositions and processes used therein) may also be used in the manufacture of a medicament for the therapeutic applications described herein.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., for use in the preparing a medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The dissolution rate of poorly water-soluble compounds may be enhanced by the use of a spray-dried dispersion, such as those described by Takeuchi, H., et al. in “Enhancement of the dissolution rate of a poorly water-soluble drug (tolbutamide) by a spray-drying solvent deposition method and disintegrants” J. Pharm. Pharmacol., 39, 769-773 (1987); and EP0901786 B1 (U.S.2002/009494), incorporated herein by reference. The compound of the present invention is typically formulated into pharmaceutical dosage forms to provide an easily controllable dosage of the drug and to give the patient an elegant and easily handleable product.

The pharmaceutical compositions also include solvates and hydrates of the compounds of the present invention. The term “solvate” refers to a molecular complex of a compound represented by Formula (I) (including pharmaceutically acceptable salts thereof) with one or more solvent molecules. Such solvent molecules are those commonly used in the pharmaceutical art, which are known to be innocuous to the recipient, e.g., water, ethanol, ethylene glycol, and the like, The term “hydrate” refers to the complex where the solvent molecule is water. The solvates and/or hydrates preferably exist in crystalline form. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The present invention further provides a method of treating diseases, conditions and/or disorders modulated by the inhibition of the acetyl-CoA carboxylases enzyme(s) in an animal that includes administering to an animal in need of such treatment a therapeutically effective amount of a compound of the present invention or a pharmaceutical composition comprising an effective amount of a compound of the present invention and a pharmaceutically acceptable excipient, diluent, or carrier. The method is particularly useful for treating diseases, conditions and/or disorders that benefit from the inhibition of acetyl-CoA carboxylases enzyme(s).

One aspect of the present invention is the treatment of obesity, and obesity-related disorders (e.g., overweight, weight gain, or weight maintenance).

Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, DC: U.S. Department of Health and Human Services, NIH publication no. 98-4083 (1998).

Another aspect of the present invention is for the treatment (e.g delaying the progression or onset) of diabetes or diabetes-related disorders including Type 1 (insulin-dependent diabetes mellitus, also referred to as “IDDM”) and Type 2 (noninsulin-dependent diabetes mellitus, also referred to as “NIDDM”) diabetes, impaired glucose tolerance, insulin resistance, hyperglycemia, and diabetic complications (such as atherosclerosis, coronary heart disease, stroke, peripheral vascular disease, nephropathy, hypertension, neuropathy, and retinopathy).

In yet another aspect of the present invention is the treatment of obesity co-morbidities, such as metabolic syndrome. Metabolic syndrome includes diseases, conditions or disorders such as dyslipidemia, hypertension, insulin resistance, diabetes (e.g., Type 2 diabetes), coronary artery disease and heart failure. For more detailed information on Metabolic Syndrome, see, e.g., Zimmet, P. Z., et al., “The Metabolic Syndrome: Perhaps an Etiologic Mystery but Far From a Myth—Where Does the International Diabetes Federation Stand?,” Diabetes & Endocrinology, 7(2), (2005); and Alberti, K. G., et al., “The Metabolic Syndrome—A New Worldwide Definition,” Lancet, 366, 1059-62 (2005). Preferably, administration of the compounds of the present invention provides a statistically significant (p<0.05) reduction in at least one cardiovascular disease risk factor, such as lowering of plasma leptin, C-reactive protein (CRP) and/or cholesterol, as compared to a vehicle control containing no drug. The administration of compounds of the present invention may also provide a statistically significant (p<0.05) reduction in glucose serum levels.

In yet another aspect of the invention is the treatment of nonalcoholic fatty liver disease (NAFLD) and hepatic insulin resistance.

For a normal adult human having a body weight of about 100 kg, a dosage in the range of from about 0.001 mg to about 10 mg per kilogram body weight is typically sufficient, preferably from about 0.01 mg/kg to about 5.0 mg/kg, more preferably from about 0.01 mg/kg to about 1 mg/kg. However, some variability in the general dosage range may be required depending upon the age and weight of the subject being treated, the intended route of administration, the particular compound being administered and the like. The determination of dosage ranges and optimal dosages for a particular patient is well within the ability of one of ordinary skill in the art having the benefit of the instant disclosure. It is also noted that the compounds of the present invention can be used in sustained release, controlled release, and delayed release formulations, which forms are also well known to one of ordinary skill in the art.

The compounds of the present invention may also be used in conjunction with other pharmaceutical agent(s) for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆(including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT to Publication No. WO 2005/116034 or U.S. Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆(as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

Suitable anti-diabetic agents include a sodium-glucose co-transporter (SGLT) inhibitor, a phosphodiesterase (PDE)-10 inhibitor, a diacylglycerol acyltransferase (DGAT) 1 or 2 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., Byetta™, exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist and a glucokinase activator. Preferred anti-diabetic agents are metformin, a glucagon-like peptide 1 (GLP-1) agonist (e.g, Byetta™) and DPP-IV inhibitors (e.g., sitagliptin, vildagliptin, alogliptin and saxagliptin).

All of the recited U.S. patents and publications (including all technical bulletins referenced in the Examples) are incorporated herein by reference in their entireties.

The Examples set forth herein below are for illustrative purposes only. The compositions, methods, and various parameters reflected herein are intended only to exemplify various aspects and embodiments of the invention, and are not intended to limit the scope of the claimed invention in any way.

### EXAMPLES

The compounds and intermediates described below were generally named according to the IUPAC (International Union for Pure and Applied Chemistry) recommendations on Nomenclature of Organic Chemistry and the CAS Index rules. Unless noted otherwise, all reactants were obtained commercially.

Flash chromatography was performed according to the method described by Still et al., J. Org. Chem., 1978, 43, 2923.

All Biotage® purifications, discussed herein, were performed using either a 40M or 40S Biotage® column containing KP-SIL silica (40-63 μM, 60 Angstroms) (Biotage AB; Uppsala, Sweden).

All CombiFlash® purifications, discussed herein, were performed using a CombiFlash® Companion system (Teledyne Isco; Lincoln, Nebr.) utilizing packed RediSep® silica columns

Mass Spectra were recorded on a Waters (Waters Corp.; Milford, Mass.) Micromass Platform II spectrometer. Unless otherwise specified, mass spectra were recorded on a Waters (Milford, Mass.) Micromass Platform II spectrometer.

Proton NMR chemical shifts are given in parts per million downfield from tetramethylsilane and were recorded on a Varian Unity 400 or 500 MHz (megaHertz) spectrometer (Varian Inc.; Palo Alto, Calif.). NMR chemical shifts are given in parts per million downfield from tetramethylsilane (for proton) or fluorotrichloromethane (for fluorine).

The preparations described below were used in the synthesis of compounds exemplified in the following examples.

### Preparation of Starting Materials and Intermediates

### Carboxylic Acid Starting Materials

The following commercially available carboxylic acids were used to prepare exemplified compounds of the present invention: 4-chloro-3-methylbenzoic acid (Alfa Aesar, Ward Hill, Mass.), 1H-pyrazolo[3,4-b]pyridine-5-carboxylic acid (Sphinx Scientific Laboratory Product List), 1-methyl-1H-indazole-6-carboxylic acid (PharmaBlock R & D Product List), 1H-benzimidazole-5-carboxylic acid (Affinitis Pharma LLC, New Haven, Conn.), 1H-indazole-5-carboxylic acid (Tyger Scientific, Inc., Ewing, N.J.), 4-amino-2-methylpyrimidine-5-carboxylic acid (Tyger Scientific, Inc., Ewing, N.J.), 2-(methylamino)isonicotinic acid (Aurora Building Blocks), 1H-pyrrolo[3,2-b]pyridine-6-carboxylic acid (Matrix Scientific), 2-methyl-1H-benzimidazole-5-carboxylic acid (Apollo Scientific Intermediates for Research and Development), 7H-pyrrolo[2,3-b]pyridine-2-carboxylic acid (Ryan Scientific Product List), 1H-pyrrolo[2,3-b]pyridine-5-carboxylic acid (Matrix Scientific), 2-oxoindoline-5-carboxylic acid (Apollo Scientific Intermediates for Research and Development), 2-oxo-2,3-dihydro-1H-benzimidazole-5-carboxylic acid (AKos Building Blocks Product List), 2-oxo-1,2,3,4-tetrahydroquinoline-7-carboxylic acid (AKos Building Blocks Product List), 2-amino-1,6-naphthyridine-3-carboxylic acid (ACES Pharma Product List), 3-aminoquinoxaline-2-carboxylic acid (AsisChem Screening Library), 7-aminopyrazolo[1,5-a]pyrimidine-6-carboxylic acid (Ryan Scientific Product List), 1-methyl-2-oxo-2,3-dihydro-1H-benzimidazole-5-carboxylic acid (AKos Building Blocks Product List), 4-(1H-imidazol-2-yl)benzoic acid (Sphinx Scientific Laboratory Product List), 3-(1H-imidazol-4-yl)benzoic acid (Apollo Scientific Intermediates for Research and Development), 5-amino-2-phenyl-2H-1,2,3-triazole-4-carboxylic acid (Ryan Scientific Screening Library), 8-methyl-4-oxo-1,4-dihydroquinoline-2-carboxylic acid (Aurora Building Blocks), 2-carbamoylnicotinic acid (J & K Scientific Product List), 8-methylimidazo[1,2-a]pyridine-2-carboxylic acid (Aurora Building Blocks), 3-(1H-pyrazol-3-yl)benzoic acid (Maybridge. Cornwall, UK), 3-(1H-pyrazol-1-yl)benzoic acid (AKos Screening Library), 1H-pyrrolo[2,3-b]pyridine-3-carboxylic acid (Aldrich), 6-morpholin-4-ylnicotinic acid (Ryan Scientific Product List), 7-methylimidazo[1,2-a]pyridine-2-carboxylic acid (Aurora Building Blocks), imidazo[1,2-a]pyridine-2-carboxylic acid (Aurora Building Blocks), 5-pyridin-3-yl-1H-pyrazole-3-carboxylic acid (AKos Screening Library), 6-methyl-2-(methylamino)nicotinic acid (Aurora Building Blocks), imidazo[1,5-a]pyridine-7-carboxylic acid (Bepharm Product List), 3H-imidazo[4,5-b]pyridine-6-carboxylic acid (Sphinx Scientific Laboratory Product List), 7-hydroxypyrazolo[1,5-a]pyrimidine-6-carboxylic acid (Butt Park Screening Library), indolizine-2-carboxylic acid (Ryan Scientific Product List), 2-pyridin-2-yl-1H-imidazole-5-carboxylic acid (Ambinter Stock Screening Collection), 3-(1H-imidazol-2-yl)benzoic acid (Greenchem Institute Product List), pyrrolo[1,2-c]pyrimidine-3-carboxylic acid (Milestone PharmTech Product List), 1H-pyrrolo[3,2-b]pyridine-2-carboxylic acid (Azasynth Building Blocks), 1H-pyrrolo[3,2-c]pyridine-2-carboxylic acid (Aurora Building Blocks), imidazo[1,2-a]pyridine-7-carboxylic acid (Bepharm

Product List), 4-(1H-1,2,4-triazol-1-yl)benzoic acid (AKos Building Blocks Product List), 1-methyl-1H-benzimidazole-5-carboxylic acid (AKos Building Blocks Product List), 6-(1H-pyrazol-1-yl)nicotinic acid (Butt Park Screening Library), 1,6-naphthyridine-2-carboxylic acid (Bepharm Product List), 1H-imidazo[4,5-b]pyridine-5-carboxylic acid (Sphinx Scientific Laboratory Product List), 1-methyl-4-oxo-4,7-dihydro-1H-pyrazolo[3,4-b]pyridine-5-carboxylic acid (Aurora Screening Library), imidazo[1,2-a]pyridine-6-carboxylic acid (Apollo Scientific Intermediates for Research and Development), 1H-pyrrolo[2,3-c]pyridine-2-carboxylic acid (Parkway Scientific Product List), 1H-indazole-6-carboxylic acid (Aldrich) quinoxaline-2-carboxylic acid (Aldrich), 3-acetamidobenzoic acid (Apollo Scientific Intermediates for Research and Development), 4-chloro-1H-indazole-6-carboxylic acid (Sinova Product List), 2-morpholinopyrimidine-5-carboxylic acid (AKos Screening Library), 1H-imidazo[1,2-b]pyrazole-6-carboxylic acid (Aurora Building Blocks), 3-hydroxyquinoline-4-carboxylic acid (AKos Screening Library), 8-hydroxyquinoline-7-carboxylic acid (TCI Laboratory Chemicals) and 3-(1H-pyrazol-4-yl)benzoic acid (AKos Building Blocks Product List).

The following carboxylic acids (which were used to prepare compounds described in the Examples below) were prepared by previously published means: 3-hydroxy-6-methylpicolinic acid (P. Korovchenko et al., Catalysis Today 2007, 121, 13-21); 4-hydroxy-1,3-dimethyl-1H-pyrazole-5-carboxylic acid (Tet. Let. 1971, 19, 1591);

3-amino-2,6-dimethylisonicotinic acid (Gulland, J. M., Robinson, R. J. Chem. Soc., Trans. 1925, 127, 1493-503); 5-hydroxyquinoline-6-carboxylic acid (Bogert, M. T.; Fisher, Harry L. Orig. Com. 8th Intern. Cangr. Appl. Chem. 1912, 6, 37-44; 5-hydroxyisoquinoline-6-carboxylic acid (can be prepared by hydrolysis of the corresponding methyl ester: Dyke, S. F.; White, A. W. C.; Hartley, D. Tetrahedron 1973, 29, 857-62); 3-methyl-1-(pyridin-3-yl)-1H-pyrazole-5-carboxylic acid (can be prepared by analogous chemistry to J. Het. Chem. 1999, 36, 217).

The following carboxylic acid starting materials (which were used to prepare compounds described in the Examples below) were prepared as described below.

Acid Preparation 1: 4-chloro-1H-benzimidazole-6-carboxylic Acid

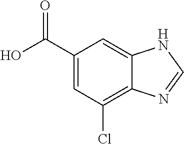

To a mixture of 4-amino-3-nitrobenzoic acid (10 g, 56 mmol) in acetic acid (100 mL) at 0° C. was added sulfuryl chloride (8.98 g, 66 mmol). The reaction mixture was allowed to warm to ambient temperature and stirred overnight. The mixture was poured into ice water, filtered and air dried to give 4-amino-5-chloro-3-nitrobenzoic (7.35 g, 62%) as yellow solid. ¹H NMR (400 MHz, DMSO-d₆) ppm 8.24 (s, 2H) 8.19 (s, 1H) 7.86 (s, 1H)

A suspension of 4-amino-5-chloro-3-nitrobenzoic (7.35 g, 34 mmol) in methanol (150 mL) was treated with concentrated sulphuric acid (40 mL). The suspension was heated to reflux overnight. The reaction solution was concentrated in vacuo to give a yellow solid which was taken up in ethyl acetate (200 mL) and water (30 mL). The solution was cooled to 0° C. and potassium carbonate was added (12.4 g) in water (30 mL). The layers were separated and the aqueous layer was extracted with ethyl acetate (200 mL). The combined organic layers were dried over magnesium sulphate and concentrated in vacuo to give methyl 4-amino-5-chloro-3-nitrobenzoate as a yellow solid (7.25 g, 93%). ¹H NMR (400 MHz, DMSO-d₆) ppm 8.50 (d, J=2.15 Hz, 1H) 8.02 (d, J=1.95 Hz, 1H) 7.84 (br. s., 2H) 3.80 (s, 3H).

To a solution of methyl 4-amino-5-chloro-3-nitrobenzoate (4.29 g, 18.6 mmol) in ethanol (115 mL), water (250 mL) and tetrahydrofuran (200 mL) was added sodium hydrosulfite (80 g, 391 mmol). The reaction was stirred at ambient temperature for two hours. To the reaction was added water (55 mL). After stirring for an additional hour saturated aqueous sodium bicarbonate (140 mL) was added to the reaction. The reaction mixture was filtered and the filtrate was extracted twice with ethyl acetate (200 mL each). The organic extracts were combined and washed with saturated aqueous sodium bicarbonate (100 mL) followed by saturated aqueous sodium chloride (100 mL). The organic layer was concentrated in vacuo to a final volume of 100 mL and then allowed to sit at ambient temperature overnight to give a precipitate. The mixture was filtered and dried under a stream of nitrogen to give methyl 3,4-diamino-5-chlorobenzoate (973 mg, 26%). The filtrate was concentrated in vacuo to give methyl 3,4-diamino-5-chlorobenzoate (2.45 g, 66%). ¹H NMR (400 MHz, DMSO-d₆) ppm 7.11 (d, J=1.95 Hz, 1H) 7.08 (d, J=1.95 Hz, 1H) 5.44 (s, 2 H) 5.08 (s, 2H) 3.70 (s, 3H).

3,4-diamino-5-chlorobenzoate (1.2 g, 6 mmol) was added to water (10 mL) and formic acid (826 mg, 18 mmol) and heated at reflux for 4 hours. The reaction was cooled to ambient temperature and aqueous potassium hydroxide was added (21 mL, 1 M). The reaction solution was washed with ethyl acetate (2×25 mL each). The aqueous layer was acidified to pH=5 with aqueous hydrochloric acid (1N) to give a precipitate which was filtered, washed with water and dried under a stream of nitrogen to give the title compound (439 mg, 37%). ¹H NMR (400 MHz, DMSO-d₆) ppm 8.45 (s, 1H) 8.10 (d, J=1.17 Hz, 1H) 7.77 (d, J=1.37 Hz, 1H).

Acid Preparation 2: 7-chloro-2-oxo-2,3-dihydro-1H-benzo[d]imidazole-5-carboxylic Acid

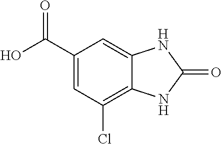

3,4-diamino-5-chlorobenzoate (from acid preparation 1, 100 mg, 0.50 mmol) and carbonyl diimidazole (89 mg, 0.55 mmol) were combined in tetrahydrofuran (2 mL) and stirred for 16 hours. The reaction solution was heated to 60° C. for 3 hours. To the reaction was added carbonyl diimidazole (81 mg, 0.50 mmol) and the reaction was continued at 60° C. for two hours. The reaction was allowed to cool to room temperature and stirred for 16 hours. A precipitate formed. The mixture was filtered. The filtrate was concentrated in vacuo and the residue was slurried in ethyl acetate. The slurry was filtered on the same filter as the original filtration. The collected solids were washed with a portion on ethyl acetate and then dried under a stream of nitrogen to give methyl 7-chloro-2-oxo-2,3-dihydro-1H-benzo[d]imidazole-5-carboxylate as a white solid (93 mg, 82%). ¹H NMR (400 MHz, DMSO-d₆) ppm 11.60 (br. s., 1H) 11.16 (s, 1H) 7.55 (d, J=1.37 Hz, 1H) 7.38 (d, J=1.56 Hz, 1H) 3.80 (s, 3H).

Methyl 7-chloro-2-oxo-2,3-dihydro-1H-benzo[d]imidazole-5-carboxylate (351 mg, 1.55 mmol), 1 M aqueous lithium hydroxide (0.774 mL, 1.55 mmol) and tetrahydrofuran (5 mL) were combined and heated to 50° C. for 2 hours. To the reaction was added 1 M aqueous lithium hydroxide (0.774 mL, 1.55 mmol) and methanol (10 mL) and the reaction was heated to reflux for 6 hours and then allowed to cool to ambient temperature overnight. The reaction solution was concentrated in vacuo to remove the tetrahydrofuran and methanol. The residual aqueous layer was extracted with ethyl acetate (2 mL). To the aqueous layer was added water (2 mL) ethyl acetate (2 mL) and 3 M aqueous hydrochloric acid. A precipitate formed. The mixture was filtered and the solids were washed with water and ethyl acetate. The solids were dried under a stream of nitrogen to give the title compound (296 mg, 90%). ¹H NMR (400 MHz, DMSO-d₆) ppm 11.54 (s, 1H) 11.12 (s, 1H) 7.54 (d, J=1.37 Hz, 1H) 7.38 (d, J=1.37 Hz, 1H).

Acid Preparation 3: 4-fluoro-1H-benzo[d]imidazole-6-carboxylic Acid

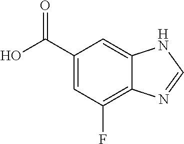

To a 2.5-5 mL microwave tube, was added 6-bromo-4-fluoro-1H-benzo[d]imidazole (160 mg, 0.744 mmol) suspended in de-gassed 1,4 dioxane (1.5 mL). To this was added trans-di(u-acetato)bis[o-(di-o-tolylphosphino)benzyl]di-palladium (II) (26 mg, 0.043 mmol) and molybdenum hexacarbonyl (100 mg, 0.38 mmol), along with sodium carbonate (237 mg, 2.23 mmol) dissolved in de-gassed water (2 mL). The mixture was stirred for 20 seconds and then heated at 155° C. in the microwave for 10 minutes, keeping the pressure under 16 bar. The vessel was vented before handling and left to stand overnight at room temperature. Water (2 mL) and ethyl acetate (3 mL) were added to the reaction, and then the mixture was filtered through Celite®. The filtrate was partitioned with ethyl acetate and separated. The aqueous fraction was washed with ethyl acetate once more and the combined organic layers were set aside. Another portion of water (5 mL) was added to the aqueous layer and acidified with 0.5 M HCl to pH 3; a brown precipitate was formed.

The mixture was allowed to stand in the refrigerator at 4° C. for 1 hour. The mixture was filtered and washed with water to give 4-fluoro-1H-benzo[d]imidazole-6-carboxylic acid as a grey solid, (63% yield). ¹H NMR (500 MHz, DMSO-d₆) ppm 12.99 (br. s., 1H) 8.47 (s, 1H) 8.02 (s, 1H) 7.52 (d, J=11.71 Hz, 1H).

Acid Preparation 4: 1-oxo-1,2-dihydroisoquinoline-6-carboxylic Acid

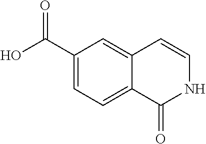

To a mixture of (E)-3-(3-bromophenyl)acrylic acid (100 g, 0.44 mol) and triethylamine (0.48 mol) in toluene (1000 mL) was added diphenylphosphoryl azide (127.4 g, 0.45 mol) dropwise at 0-10° C. The mixture was stirred at room temperature overnight. Thin layer chromatography (petroleum ether/ethyl acetate=8:1) indicated completion of reaction. The resulting mixture was washed with 1 N sodium hydroxide (500 mL) and extracted with ethyl acetate (2000 mL×3). The organic layer was concentrated to give crude (E)-1-azido-3-(3-bromophenyl)prop-2-en-1-one, which was used in the next step directly.

A mixture of crude (E)-1-azido-3-(3-bromophenyl)prop-2-en-1-one (crude about 120 g) and toluene (200 mL) was refluxed for two hours. Thin layer chromatography (petroleum ether/ethyl acetate=8:1) indicated most of the starting material was consumed. The mixture was concentrated to give crude (E)-1-bromo-3-(2-isocyanatovinyl)benzene (100 g, 94%), which was used in the next step directly.

A solution of (E)-1-bromo-3-(2-isocyanatovinyl)benzene (100 g, 0.44 mol) in toluene (200 mL) was added dropwise to a mixture of tributylamine (100 mL) and oxydibenzene (500 mL) at 190° C. After the addition, the mixture was heated at 210° C. for another two hours. Thin layer chromatography (petroleum ether/ethyl acetate=1:1) indicated the reaction was complete. The mixture was cooled to room temperature, filtered, and the solid was washed with ethyl acetate (50 mL×3). The solid was dried under vacuum to give crude 6-bromoisoquinolin-1(2H)-one (30 g, 30%) as a light yellow solid, which was used in the next step directly.

A mixture of 6-bromoisoquinolin-1(2H)-one (30 g, 134 mmol), triethylamine (17.6 g,174 mmol), palladium (II) chloride (0.24 g, 1.34 mmol) and (S)-(−)-2,2′-bis(diphenylphosphino)-1,1′-binaphthyl (0.84 g, 1.34 mmol) in methane (300 mL) was heated at 100° C. under 2 MPa of carbon monoxide and stirred for 12 hours. Thin layer chromatography (petroleum ether/ethyl acetate=1:1) indicated the reaction was complete. The reaction mixture was concentrated, the residue was washed with water, and the solid was filtered and dried in vacuum to give crude methyl 1-oxo-1,2-dihydroisoquinoline-6-carboxylate (23.8 g, 95%) as a yellow solid, which was used in the next step directly.

To a mixture of methyl 1-oxo-1,2-dihydroisoquinoline-6-carboxylate (25 g, 0.133 mol), tetrahydrofuran (200 mL) and water (200 mL) was added lithium hydroxide (16.8 g, 0.40 mol) at room temperature, and the mixture was stirred for four hours. Thin layer chromatography (petroleum ether/ethyl acetate=1:1) indicated the reaction was complete. The reaction mixture was extracted with ethyl acetate (100 mL×3) to remove impurities. The aqueous layer was acidified with 4 N aqueous HCl to pH 5 and filtered. The solid was dried in vacuum to give 1-oxo-1,2-dihydroisoquinoline-6-carboxylic acid (11.3 g, 48%) as a light yellow solid. ¹H NMR (400 MHz, DMSO-d₆) ppm 11.48 (s, 1H), 8.24 (d, 2H), 7.93 (d, 1H), 7.22 (d, 1H), 6.68 (d, 1H).

Acid Preparation 5: 1-oxo-1,2-dihydroisoquinoline-7-carboxylic Acid

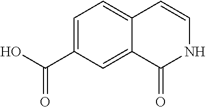

1-oxo-1,2-dihydroisoquinoline-7-carboxylic acid was prepared in an analogous fashion to 1-oxo-1,2-dihydroisoquinoline-6-carboxylic acid, (acid preparation 4).

Acid Preparation 6: 5-(1H-imidazol-1-yl)picolinic Acid

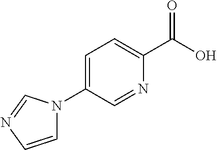

5-bromopicolinonitrile (2.0 g, 10.9 mmol), imidazole (818 mg, 12 mmol) potassium carbonate (1.66 g, 12 mmol) and dimethylformamide (40 mL) were combined and heated to 130° C. for 20 hours. The reaction solution was evaporated and the residue was partitioned between dichloromethane (150 mL) and water (100 mL). The phases were separated and the organic phase was washed with water (50 mL), dried over magnesium sulfate and evaporated to give a residue which was purified by flash chromatography eluting with 2-3% methanol in dichloromethane gradient to give 5-(1H-imidazol-1-yl)picolinonitrile (1.23 g, 66%).

5-(1H-imidazol-1-yl)picolinonitrile (136 mg, 0.80 mmol) was heated to reflux in 6N aqueous hydrochloric acid (10 mL) for 2 hours. The reaction mixture was evaporated and the residue was azeotroped with three portions of toluene to give a residue which was purified on an ion exchange column (AG-50 Biorad) eluting with a 0-10% pyridine in water gradient to give the title compound as a white solid (128 mg, 84%). ¹H NMR (400 MHz, DMSO-d₆) ppm 9.10 (s, 1H), 8.50 (s, 1H), 8.26-8.33 (m, 1H), 8.13-8.20 (m, 1H), 7.96 (s, 1H), 7.16 (s, 1H).

Acid Preparation 7: 7-chloro-1H-indazole-5-carboxylic Acid

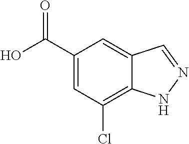

To a mixture of 4-amino-3-chloro-5-methyl-benzonitrile (3.0 g, 18.0 mmol) in chloroform (50 mL) was added acetic anhydride (3.92 mL, 41.4 mmol). The mixture was heated at reflux for 5 hours and then cooled to room temperature. To the mixture was added potassium acetate (530 mg, 5.4 mmol) and isoamyl nitrite (5.28 mL, 39.6 mmol). The reaction was heated at reflux for 16 hours. The reaction mixture was cooled to room temperature, extracted with saturated aqueous sodium bicarbonate, the organics were dried over sodium sulfate, and concentrated in vacuo to afford a brown oil. The oil was dissolved in methanol (25 mL) and concentrated hydrochloric acid (25 mL) was added. The reaction was stirred at room temperature for 22 hours and the methanol was concentrated in vacuo. The remaining aqueous layer was adjusted to a pH of 7 and the resultant precipitate was filtered to afford a brown solid which was purified by flash chromatography using 50% dichloromethane in heptane as eluent to afford 7-chloro-1H-indazole-5-carbonitrile as a solid (585 mg, 18%): −ESI MS (M−1) 176.0; ¹H NMR (400 MHz, CDCl₃) ppm 8.29 (br. s., 2H), 8.08 (s, 1H), 7.61 (s, 1H).

To a mixture of 7-chloro-1H-indazole-5-carbonitrile (1.36 g, 7.66 mmol) in ethanol (52.5 mL) was added water (17.5 mL) and potassium hydroxide (6.44 g, 115 mmol). The reaction mixture was heated at reflux for 16 hours. The reaction mixture was cooled to room temperature, extracted twice with ethyl ether, acidified the aqueous with 1N hydrochloric acid and the resultant precipitate was filtered to afford 7-chloro-1H-indazole-5-carboxylic acid as a brown solid (900 mg, 60%): −ESI MS (M−H) 195.2.

Acid Preparation 8: 5-morpholinopicolinic Acid

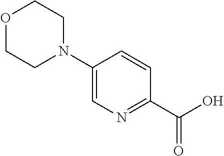

Diethyl malonate (151 g, 0.944 mol) was added dropwise under stirring to 60% sodium hydride in mineral oil (37.8 g, 0.944 mol) in dry tetrahydrofuran (1 L). After hydrogen evolution ceased, 2-chloro-5-nitropyridine (125 g, 0.787 mol) was added. The reaction mixture was refluxed for 2 hours and then the tetrahydrofuran was evaporated in vacuo to give crude diethyl (5-nitropyridin-2-yl)malonate, which was used at the next stage without purification.

Crude diethyl (5-nitropyridin-2-yl)malonate was added to boiling 65% nitric acid (1.5 L) under stirring. The reaction mixture was refluxed under stirring for 15 hours. The reaction mixture was concentrated in vacuo and the resulting solid was washed with chloroform to give 5-nitropyridine-2-carboxylic acid (yield 65%, 85.9 g).

5-Nitropyridine-2-carboxylic acid (100 g, 0.60 mol) was heated at reflux in methanol (1 L) and sulfuric acid (57 mL) for 5 hours. The reaction mixture was cooled, reduced to half volume in vacuo, and the residue neutralized with a solution of sodium carbonate. The resulting precipitate was filtered to give methyl 5-nitropyridine-2-carboxylate (yield 89%, 98 g).

Methyl 5-nitropyridine-2-carboxylate (182 g, 1 mol) was refluxed in piperidine (250 mL) for 1 hour. The reaction mixture was concentrated in vacuo to give crude 5-nitro-2-(piperidin-1-ylcarbonyl)pyridine, which was used for the next stage without additional purification.

Crude 5-nitro-2-(piperidin-1-ylcarbonyl)pyridine was reduced by hydrogen under atmospheric pressure in the presence of 10% palladium on carbon (4 g) in acetic acid (500 mL). The catalyst was separated by filtration and the solvent evaporated in vacuum to give crude 6-(piperidin-1-ylcarbonyl)pyridin-3-amine, which was used for the next stage without additional purification.

A solution of sodium nitrite (69 g) in concentrated hydrochloric acid (1.5 L) was added to crude 6-(piperidin-1-ylcarbonyl)pyridin-3-amine at 0 C, and the mixture was stirred for 10 minutes. Urea (20 g) was added, and the mixture was stirred for 15 minutes. Sodium iodide (150 g) was added, and the product was separated by filtration and recrystallized from ethanol to give 5-iodo-2-(piperidin-1-ylcarbonyl)pyridine (yield 23% calculated for methyl 5-nitropyridine-2-carboxylate, 71 g.

A mixture of 5-iodo-2-(piperidin-1-ylcarbonyl)pyridine (71 g, 0.23 mol), palladium (II) acetate (1.03 g, 46 mmol), 2-(di-tert-butylphosphino)biphenyl (2.76 g, 92 mmol), morpholine (23.7 g, 0.28 mol), and sodium tert-butoxide (27.8 g, 0.28 mol) in toluene (400 mL) was stirred under argon at 95 C for 2 hours. The product was isolated by chromatography (silica gel, ethyl acetate) and recrystallized from ethanol to give 4-[6-(piperidin-1-ylcarbonyl)pyridin-3-yl]morpholine (yield 37%, 22 g).

25% KOH (100 mL) was added to 4-[6-(piperidin-1-ylcarbonyl)pyridin-3-yl]morpholine (18.3 g), and the mixture was refluxed and then neutralized with HCl. The solution was evaporated in vacuum, and the product was extracted with hot isopropanol to give the title compound (yield 71%, 11.5 g). +ESI MS (M+H) 209.7; ¹H NMR (400 MHz, DMSO-d₆) ppm 8.34 (br. s., 1H), 8.03 (d, 1H), 7.65 (dd, 1H), 3.75 (br. s., 4H), 3.40 (br. s., 4H).

Acid Preparation 9: 7-chloro-2-methyl-1H-benzo[d]imidazole-5-carboxylic Acid

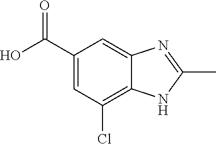

Add 2N hydrochloric acid (8 mL) to a solution of 3,4-diamino-5-chlorobenzoate (from acid preparation 1, 435 mg, 2.17 mmol) in ethanol (20 mL). Heat the mixture to reflux then add acetylacetone (437 mg, 4.37 mmol) to the yellow solution. The yellow solution turned purple upon addition. Stir at reflux for 1 hour and the solution turned back to yellow. Stir at reflux for an additional 1 hour. Concentrate the solvent to a colorless residue. Add water (20 mL). Extract the suspension with ethyl acetate (20 mL). Basify the aqueous layer with 2N sodium hydroxide (˜8 mL) to pH˜10. Extract with ethyl acetate (3×15 mL). Wash combined organics from the basic extraction with brine (5 mL). Dry over magnesium sulfate, filter, concentrate, and dry under high vacuum to yield methyl 7-chloro-2-methyl-1H-benzo[d]imidazole-5-carboxylate (290 mg, 59%) as a colorless solid. ¹H NMR (400 MHz, CDCl₃) ppm 2.68 (s, 3H), 3.93 (s, 3H), 7.25 (s, 1H), 7.96 (s, 1H).

Add 2N sodium hydroxide (5 mL, 5 mmol) to a solution of methyl 7-chloro-2-methyl-1H-benzo[d]imidazole-5-carboxylate (280 mg, 1.25 mmol) in methanol (7.5 mL). Stir at 65° C. for 16 hours. The methanol was concentrated in vacuo and the remaining aqueous layer was extracted with ethyl acetate (10 mL). Acidify the aqueous layer to pH ˜4 with 1N hydrochloric acid (˜5 mL). A colorless precipitate was filtered and dried under high vacuum to yield the title compound (189 mg, 72%). ¹H NMR (400 MHz, CD₃OD) ppm 2.61 (s, 3H), 7.86 (d, J=1.37 Hz, 1H), 8.08 (d, J=1.17 Hz, 1H).

Acid Preparation 10: 7-chloro-2-methyl-1H-benzo[d]imidazole-5-carboxylic Acid

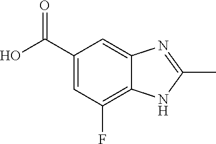

A round bottomed flask was charged with 5-bromo-3-fluorobenzene-1,2-diamine (400 mg, 2 mmol) and 30 mL ethanol. 5 N hydrochloric acid (8 mL, 40 mmol) was then added. This mixture was heated to reflux and 2,4-pentanedione was added. The reaction mixture turned deep purple in color then slowly turned back to tan. Reaction was allowed to proceed for 3 hours and then cooled and neutralized with saturated sodium bicarbonate solution. The reaction mixture was then extracted three times with dichloromethane. The combined organic layers were washed with brine, dried with magnesium sulfate, filtered and concentrated in vacuo. The crude mixture was triturated in diethyl ether then filtered to give 6-bromo-4-fluoro-2-methyl-1H-benzo[d]imidazole (375 mg, 82%) as a tan solid. ¹H NMR (400 MHz, CDCl₃) ppm 7.44 (br. s., 1H), 7.10 (d, J=11.22 Hz, 1H), 2.63 (s, 3H).

A 5 mL microwave vial was charged 6-bromo-4-fluoro-2-methyl-1H-benzo[d]imidazole (187 mg, 0.815 mmol) and suspended in de-gassed dioxane (2 mL), trans-di- -acetatobis[2-(di-O-tolylphosphino)benzyl]dipalladium (II) (28 mg, 0.048 mmol) and molybdenumhexacarbonyl (110 mg, 0.417 mmol). Degassed 10% aqueous sodium carbonate (2.45 mL, 2.45 mmol) was then added. The reaction was then stirred for 20 seconds before being reacted in the microwave at 155° C. at very high absorption for 10 minutes. The vessel was then vented and left to stand overnight at room temperature. Water (2mL) and ethyl acetate (3 mL) were then added and the mixture was filtered through Celite®. The layers were separated and the aqueous layer was washed with ethyl acetate (×2). The combined ethyl acetate layers were set aside. Water (5 mL) was added to the aqueous layer which was then acidified with 0.5 M hydrochloric acid to a pH of 3 then cooled to 4° C. A solid formed which was filtered and washed with water to give the title compound (61 mg, 37%) as a yellow solid. A second crop formed which was then filtered to give the title compound (100 mg, 63%). ¹H NMR (500 MHz, CD₃OD) ppm 8.22 (d, J=0.98 Hz, 1H), 7.91 (d, J=10.49 Hz, 1H), 2.95 (s, 3H).

Acid Preparation 11: 1H-pyrazolo[4,3-b]pyridine-6-carboxylic Acid

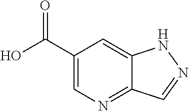

To a suspension of sodium hydride (5.08 g, 127 mmol) in dimethylformamide (75 mL) was added diethyl malonate (19.26 mL, 127 mmol) at 0° C. The solution was then stirred at ambient temperature for 30 minutes and a solution of 5-bromo-2-chloro-3-nitropyridine (30 g, 127 mmol) in dimethylformamide (75 mL) was added dropwise. The dark brown mixture was then stirred at 100° C. for 2 hours before being cooled to ambient temperature and quenched with a saturated solution of ammonium chloride (500 mL) at 0° C. The mixture was extracted with ethyl acetate (3×500 mL) and the combined organics were dried over magnesium sulfate. The solvent was removed in vacuo to give a dark brown oil which was purified by flash column chromatography (10% ethyl acetate/hexane) to afford diethyl 2-(5-bromo-3-nitropyridin-2-yl)malonate as a brown solid (31.8 g, 88 mmol, 69%). ¹HNMR (400 MHz, CDCl₃): ppm 8.86 (s, 1H), 8.61 (s, 1H), 5.44 (1H, s), 4.29 (q, 4H), 1.27 (t, 6H).

A mixture of the diethyl 2-(5-bromo-3-nitropyridin-2-yl)malonate (31.8 g, 88 mmol) in aqueous hydrochloric acid (6M, 1.4 L) was stirred at reflux for 18 hours. The reaction mixture was cooled to ambient temperature and added very slowly to a saturated aqueous solution of aqueous sodium bicarbonate (4 L) at 0° C. The mixture was then extracted with dichloromethane (7 L), dried over magnesium sulfate and the solvent removed in vacuo to give 5-bromo-2-methyl-3-nitropyridine as an orange oil (13.8 g, 63.9 mmol, 72%) which solidified upon standing. ¹HNMR (300 MHz, CDCl₃): ppm 8.78 (s, 1H), 8.43 (s, 1H), 2.79 (s, 3H).

To a solution of 5-bromo-2-methyl-3-nitropyridine (13.8 g, 63.9 mmol) in industrial methylated spirit (330 mL) at 40° C. was added iron powder (20 g) (portionwise to avoid clumping) followed by concentrated aqueous hydrochloric acid (5 mL). The dark brown mixture was stirred vigorously at reflux for 2 hours and then cooled and filtered through Celite® (which was washed with 1 L of industrial methylated spirit). The solvent was then removed in vacuo and the residue taken up in ethyl acetate (200 mL) and washed with a saturated aqueous solution of sodium bicarbonate (200 mL), dried over magnesium sulfate and solvent removed in vacuo to give 5-bromo-2-methylpyridin-3-amine as an orange solid, (10.7 g, 57.5 mmol, 89.9%). ¹HNMR (400 MHz, CDCl₃): ppm 7.91 (s, 1H), 7.00 (s, 1H), 3.75 (br.s, 2H), 2.25 (s, 3H).

To a solution of 5-bromo-2-methylpyridin-3-amine (10.7 g, 57.5 mmol) in dichloromethane (575 mL) was added acetic anhydride (12 mL, 126.5 mmol) at 0° C. followed by triethylamine (22 mL, 158 mmol). The mixture was allowed to warm to ambient temperature and stirred for 18 hours at which point a further equivalent of acetic anhydride (6 mL, 63 mmol) was added. The mixture was stirred at ambient temperature for a further 72 hours. The reaction mixture was quenched with a saturated aqueous solution of sodium bicarbonate (500 mL) and the organic phase washed with saturated aqueous sodium chloride (500 mL), dried over magnesium sulfate and concentrated in vacuo to give a brown solid. This solid was triturated with 30% ethyl acetate in hexanes to yield N-(5-bromo-2-methylpyridin-3-yl)acetamide as an off-white solid, (8.28 g, 36 mmol, 63%). ¹HNMR (400 MHz, CD₃OD): ppm 8.31 (s, 1H), 8.18 (s, 1H), 2.43 (s, 3H), 2.18 (s, 3H).

To a solution of N-(5-bromo-2-methylpyridin-3-yl)acetamide (8.28 g, 36 mmol) in chloroform (550 mL) at ambient temperature was added potassium acetate (4.32 g, 43.6 mmol), acetic acid (2.5 mL, 43.6 mmol) and followed by acetic anhydride (6.86 mL, 72.6 mmol). The mixture was stirred at ambient temperature for 15 minutes before being heated to 40° C. Isoamylnitrite was then added dropwise. The reaction was then stirred at 60° C. for 48 hours. The reaction mixture was poured slowly into a saturated solution of sodium bicarbonate (500 mL) at 0° C. The organic phase was retained and the aqueous phase extracted with dichloromethane (500 mL). The combined organics were then concentrated to a brown oil which was dissolved in methanol (500 mL). Aqueous sodium hydroxide (2 M, 500 mL) was added at 0° C. and the mixture stirred at ambient temperature for 1 hour before the methanol was removed in vacuo. The aqueous mixture was then extracted with ethyl acetate (3×500 mL). The combined organics dried over magnesium sulfate, and the solvent removed in vacuo to give 6-bromo-1H-pyrazolo[4,3-b]pyridine as a light brown solid (5.5 g, 27.9 mmol, 77%). ¹HNMR (400, CD₃OD): ppm 8.55 (s, 1H), 8.24 (s, 1H), 8.21 (s, 1H).

To a solution of 6-bromo-1H-pyrazolo[4,3-b]pyridine (5.5 g, 27.9 mmol) in methanol (125 mL) and acetonitrile (75 mL) was added triethylamine (22 mL, 156 mmol), 2,2′-bis(diphenylphosphino)-1,1′-binaphthyl (1.98 g, 3.07 mmol), palladium dichloride (1.23 g, 6.98 mmol). The mixture was placed under 20 bar of carbon monoxide, heated to 100° C., and stirred vigorously for 18 hours. The reaction mixture was cooled to ambient temperature and filtered through Celite® before the solvent was removed in vacuo to yield a brown oil. This crude oil was then purified by flash column chromatography (1:1, ethyl acetate:hexane) to give methyl 1H-pyrazolo[4,3-b]pyridine-6-carboxylate as a pale yellow solid (4.52 g, 92% yield). ¹HNMR (400, CDCl₃) ppm 10.56 (s, 1H), 9.23 (s, 1H), 8.35 (s, 1H), 8.40 (s, 1H), 4.01 (s, 3H).

To a solution of methyl 1H-pyrazolo[4,3-b]pyridine-6-carboxylate (3.52 g, 20 mmol) in methanol (250 mL) and water (190 mL) at 0° C. was added aqueous sodium hydroxide (2M, 64 mL, 128 mmol), dropwise. The suspension was then allowed to warm to ambient temperature and stirred for 18 hours. The methanol was then removed in vacuo and the aqueous mixture extracted with ethyl acetate (250 mL) before being acidified (to pH 5-6) with aqueous hydrochloric acid (2 M, 70 mL). The cream solid which had precipitated out was then filtered off and dried in a desiccator to yield the title compound (0.675 g, 4.16 mmol, 21% yield). ¹HNMR (400 MHz, DMSO-d₆): ppm 8.97 (s, 1H), 8.45 (s, 1H), 8.39 (s, 1H).

Acid Preparation 12: 3-cyano-1H-indazole-5-carboxylic Acid

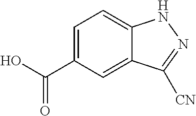

A suspension of (2-nitrophenyl)-acetonitrile (30 g, 185 mmol) and 10% palladium on carbon (2 g) in acetic acid (450 mL) was hydrogenated in a Parr apparatus under 30 psi pressure at ambient temperature for 2 hours. The mixture was filtered through a Celite® pad and the filtrate was concentrated in vacuo. The obtained residue was dissolved in ethyl acetate (250 mL). The resulting solution was washed with water (2×100 mL) and saturated sodium chloride (50 mL), and then dried over anhydrous sodium sulfate and concentrated in vacuo to yield product. The crude material was purified by column chromatography (100-200 mesh silica gel) using 8% ethyl acetate in petroleum ether as eluent to afford (2-aminophenyl)acetonitrile (13.5 g, 55%) as a solid. ¹HNMR (CDCl₃) ppm 7.3-7.1 (m, 2H), 6.9-6.7 (m, 2H), 3.7 (br, 2H), 3.5 (s, 2H).

To a cooled solution of (2-aminophenyl)acetonitrile (13 g, 98 mmol) in dimethylformamide (150 mL) at 0° C., was added N-bromosuccinimide (19.3 g, 108 mmol) in portions for 30 minutes and maintained at 0° C. for 1 hour. The mixture was diluted with ethyl acetate (300 mL) and washed with water (3×100 mL) and saturated sodium chloride (50 mL). The organic layer was dried over anhydrous sodium sulfate and concentrated in vacuo. The obtained crude product was purified by column chromatography (100-200 mesh silica gel) using 10% ethyl acetate in petroleum ether as eluent to afford (2-amino-5-bromophenyl)acetonitrile (11 g, 53%) as solid. ¹HNMR (CDCl₃) 7.35 (s, 1H), 7.25(d, 1H), 6.65(d, 1H), 3.7 (br, 2H), 3.52(s, 2H).

To a cooled solution of (2-amino-5-bromophenyl)acetonitrile (11 g, 52 mmol) in concentrated hydrochloric acid (110 mL) at −50° C., a solution of sodium nitrite (3.9 g, 57 mmol) in water (20 mL) was added slowly. After the addition, the mixture was stirred at −50° C. for 2h. The mixture was neutralized with 33% ammonium hydroxide at 0° C. and extracted with ethyl acetate (3×100 mL). The combined organic layers were washed with saturated sodium chloride (100 mL), dried over anhydrous sodium sulfate and concentrated. The obtained crude product was purified by column chromatography (100-200 mesh silica gel) using 10% ethyl acetate in petroleum ether as eluent to afford 5-bromo-3-cyanoindazole (7 g, 60%) as a solid. ¹HNMR (CDCl₃) ppm 10.7 (br, 1H), 8.1 (s, 1H), 7.64 (d, 1H), 7.5 (d, 1H).

A suspension of 5-bromo-3-cyanoindazole (3 g, 13.51 mmol), palladium dichloride 1,1′-bis(diphenylphosphino)ferrocene (1.76 g, 2.16 mmol), sodium acetate (3.32 g, 40.5 mmol), dimethylformamide (1 mL) in methanol (100 mL) was degassed, and kept under carbon monoxide (80 psi) pressure at 80° C. in a autoclave for 16 hours. The mixture was diluted with water (50 mL), filtered through Celite® bed and the filtrate was concentrated. The obtained residue was acidified with 10% citric acid solution and extracted with ethyl acetate (2×100 mL). The combined organic layers were washed with brine (50 mL), dried over anhydrous sodium sulfate and concentrated. The obtained crude product was purified by column chromatography (100-200 mesh silica gel) using 10% ethyl acetate in chloroform as eluent to afford methyl 3-cyano-1H-indazole-5-carboxylate (1.8 g, 68%) as a solid. ¹HNMR (CDCl₃) ppm 10.8 (s, 1H), 8.7 (s, 1H), 8.22 (d, 1H), 7.64 (d, 1H), 4.0 (s, 3H).

To a solution of methyl 3-cyano-1H-indazole-5-carboxylate (2.5 g, 12 mmol) in ethanol (40 mL), a solution of lithium hydroxide (1.04 g, 24.9 mmol) in water (15 mL) was added and stirred at ambient temperature for 16 h. The mixture was concentrated and the obtained residue was dissolved in water (25 mL) and washed with ethyl acetate (20 mL). The aqueous layer was acidified with 10% citric acid solution, the obtained precipitate was filtered, washed with 50% ethyl acetate in petroleum ether (2×10 mL) and dried to afford the title compound (1.9 g, 82%) as a brown solid. ¹HNMR (DMSO-d₆) ppm 13.8-12.4 (br, 2H), 8.44 (s, 1H), 8.1 (d, 1H), 7.82 (d, 1H).

Acid Preparation 13: 2-(1H-pyrazol-3-yl)isonicotinic Acid

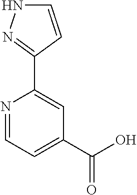

To a stirred solution of 29.0 g (69 mmol) 2-bromo-4-methylpyridine in 150 mL concentrated sulfuric acid was added portionwise 67.9 g (231 mmol) potassium dichromate. The reaction mixture was cooled with an ice bath so that the temperature stayed between 20-50° C. After the addition was complete, stirring was continued at room temperature for a further 2 hours. The reaction mixture was then poured slowly onto 2 L ice-water and the mixture stirred for 1 hour at room temperature. The resulting crystals were collected by filtration, washed with water until the washings were colorless, and dried in vacuo to afford 30.0 g (88%) of 2-bromoisonicotinic acid.

To an ice cooled solution of 2-bromoisonicotinic acid (73 g, 0.361 mol) in dichloromethane (500 mL) and methanol (35 g, 1.08 mol) was added 1-ethyl-3-[3-(dimethylamino)propyl]carbodiimide hydrochloride (67 g, 0.434 mol) by portions. The mixture was stirred at ambient temperature overnight. Then 120 g silica gel was added and the solvent evaporated. The residue was purified by flash chromatography, eluting with 5% ethyl acetate in petroleum ether to afford 58 g (75%) of methyl 2-bromoisonicotinate as a white solid.

Methyl 2-bromoisonicotinate (216 g, 1 mol), dry acetonitrile (1.7 L), ethynyl(trimethyl)silane (117 g, 1.2 mol), diisopropylamine (122 g, 1.2 mol), and dichlorobis(triphenylphosphine)palladium (36 g, 0.05 mol) were placed into a well dried three necked flask which was twice purged with a stream of nitrogen. The reaction mixture was stirred for 0.5 hours, cooled to 10° C., and copper iodide (19 g, 0.1 mol) was added under a stream of nitrogen. At 20° C., the reaction mixture became thick and black and an exotherm was observed which was followed by formation of a precipitate. After the addition of copper iodide, the reaction mixture was stirred for further 2 hours at ambient temperature. The precipitated residue was separated by filtration and twice washed with diethyl ether (800 mL). The filtrate was washed with saturated ammonium chloride (2×300 mL) and brine (2×300 mL). After drying over sodium sulfate, the solvent was evaporated. The residue was purified using a silica gel column, eluting with hexane followed by 5% ethyl acetate in petroleum ether to yield 191 g (82%) of methyl 2-((trimethylsilyl)ethynyl)isonicotinate.

Concentrated sulfuric acid (60 mL, 1.1 mol) was added to a suspension of methyl 2-((trimethylsilyl)ethynyl)isonicotinate (127 g, 0.54 mol) in tetrahydrofuran (600 mL) and mercury acetate (51.5 g, 0.16 mol). The suspension was stirred for 3 hours at 50° C. and kept overnight. The reaction mixture was diluted with diethyl ether (1.5 L) and the sulfuric acid was neutralized with saturated sodium bicarbonate (150 g, 1.7 mol). A residue of mercury salts was separated by filtration. The ether solution was washed with water and dried over sodium sulfate. The solvent was removed to give methyl 2-acetylisonicotinate as an oil that was directly used in the next step.

To a 2 L three necked flask was added methyl 2-acetylisonicotinate (160 g, 0.894 mol), dimethylformamide-dimethylacetamide (350 mL) and toluene (350 mL). The mixture was refluxed for about 5 hours with a Dean-Stark trap to remove methanol produced. Additional dimethylformamide-dimethylacetamide and toluene was added to keep the reaction volume at about 800-900 mL. When liquid chromatography-mass spectrometry showed reaction completed, the solvent was removed to yield crude (Z)-methyl 2-(3-(dimethylamino)acryloyl)isonicotinate as a dark solid. The crude solid was directly used in the next step.

To a 2 L three-necked flask was added (Z)-methyl 2-(3-(dimethylamino)acryloyl)isonicotinate (0.894 mol), hydrazine hydrate (48.8 g), anhydrous ethanol (1 L). The suspension was stirred at 20° C. overnight. The solvent was removed in vacuo. The residue was taken up in concentrated hydrochloric acid (600 mL) and heated to reflux for 2 hours. The mixture was cooled to ambient temperature. The resultant precipitate was filtered, washed with water, ethanol and acetone and dried to give 78.6 g of the title compound as a brown solid. ¹HNMR (DMSO-d₆/D₂O) ppm 8.80 (d, 1H), 8.50 (s, 1H), 7.91 (d, 1H), 7.87 (dd, 1H), 7.15 (d, 1H).

Acid Preparation 14: 3-methyl-2-oxo-2,3-dihydro-1H-benzo[d]imidazole-5-carboxylic Acid

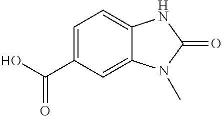

Add an aqueous solution of sodium hydrosulfite (17.4 g, 100 mmol in 80 mL of water) to methyl-3-(methylamino)-4-nitrobenzene carboxylate (855 mg, 4.75 mmol) in tetrahydrofuran (70 mL) and ethanol (30 mL) at 0° C. The orange solution turned to an orange suspension upon addition. Stir the mixture at room temperature for 2 hours. The orange suspension turned to a yellow suspension over this time. Add saturated sodium bicarbonate (100 mL) then the yellow suspension turned colorless. Extract the mixture with ethyl acetate (2×100 mL). Wash the combined organics with brine (30 mL). Dry over magnesium sulfate, filter, concentrate, and dry under high vacuum to yield methyl 4-amino-3-(methylamino)benzoate (586 mg, 68%) as a yellow oil. The resulting oil began to crystallize upon standing after 10 minutes. ¹H NMR (400 MHz, CDCl₃) ppm 2.89 (s, 3H), 3.37-3.81 (m, 2H), 3.85 (s, 3H), 6.67 (d, J=8.01 Hz, 1H), 7.33 (d, J=1.37 Hz, 1H), 7.44 (dd, J=8.11, 1.66 Hz, 1H).

Add carbonyl diimidazole (567 mg, 3.50 mmol) to a solution of methyl 4-amino-3-(methylamino)benzoate (586 mg, 3.19 mmol) in tetrahydrofuran (20 mL) at room temperature. Stir the yellow solution at room temperature for 16 hours. Add carbonyl diimidazole (500 mg, 0.96). Stir at room temperature for 4 hours. Add ethyl acetate (75 mL). Wash with 10% citric acid (5 mL), 1N sodium hydroxide (5 mL), and brine (5 mL). Dry the organics over magnesium sulfate, filter, concentrate, and dry under high vacuum to yield a crude yellow solid (690 mg, 100%). Triturate this crude solid with ethyl acetate (10 mL). Filter the precipitate and dry under high vacuum to yield methyl 3-methyl-2-oxo-2,3-dihydro-1H-benzo[d]imidazole-5-carboxylate (422 mg, 64%). ¹H NMR (400 MHz, CDCl₃) ppm 3.46 (s, 3H), 3.92 (s, 3H), 7.12 (d, J=8.21 Hz, 1H), 7.68 (s, 1H), 7.84 (dd, J=8.31, 1.47 Hz, 1H), 9.87-10.03 (m, 1H).

Add 1N sodium hydroxide (6.1 mL, 6.1 mmol) to a suspension of methyl 3-methyl-2-oxo-2,3-dihydro-1H-benzo[d]imidazole-5-carboxylate (420 mg, 2.04 mmol) in methanol (10 mL). The suspension turned to a solution upon addition of 1N sodium hydroxide. Stir at 65° C. for 16 hours. Cool to room temperature then concentrate to remove the methanol. Extract the aqueous with ethyl acetate (5 mL). Acidify the aqueous with 2N hydrogen chloride (3 mL) to pH˜2. Concentrate the aqueous layer to a solid. Triturate the solid with water (3 mL). Filter the precipitate and dry under high vacuum to yield the title compound (234 mg, 59%) as a pale brown solid. ¹H NMR (400 MHz, DMSO-d₆) ppm 3.28 (s, 3H), 7.01 (d, J=8.21 Hz, 1H), 7.57 (s, 1H), 7.63 (dd, J=8.11, 1.27 Hz, 1H), 11.19 (s, 1H), 12.60 (s, 1H).

Acid Preparation 15: 7-bromo-2-oxo-2,3-dihydro-1H-benzo[d]imidazole-5-carboxylic Acid

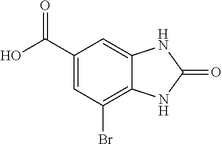

A suspension of methyl 4-amino-3-bromo-5-nitrobenzoate (10 g, 36.3 mmol) and tin(II) chloride (33 g, 14.5 mmol) in methanol (100 mL) was heated to 60° C. and maintained for 4 hours. The reaction mass was cooled to ambient temperature and concentrated to obtain a residue; the residue was basified using saturated aqueous sodium bicarbonate until pH was 11 and the aqueous layer was extracted with dichloromethane (3×200 mL). The combined organic layers were washed with aqueous saturated sodium chloride (200 mL), dried over anhydrous sodium sulfate and concentrated to obtain methyl 3,4-diamino-5-bromobenzoate as an off-white solid (5 g, 58%). ¹HNMR (CDCl₃): ppm 7.74 (s, 1H), 7.35 (s, 1H), 4.18 (broad s, 2H), 3.85 (s, 3H) and 3.38-3.56 (broad s, 2H).

A solution of 3,4-diamino-5-bromobenzoate (1 g, 4.0 mmol) and triethylamine (0.4 g, 4.0 mmol) in dichloromethane (6 mL) was cooled to 0° C. A solution of triphosgene (1.2 g, 4.08 mmol) in dichloromethane (15 mL) was added to this solution. The reaction mixture was allowed to warm to ambient temperature and maintained for 18 hours. The reaction mass was quenched with water (3 mL) and extracted with ethyl acetate (3×10 mL). The combined organic layers were washed with aqueous saturated sodium chloride (50 mL), dried over anhydrous sodium sulfate and concentrated to obtain methyl 7-bromo-2-oxo-2,3-dihydro-1H-benzo[d]imidazole-5-carboxylate as an off-white solid (500 mg, 45%). ¹HNMR (CDCl₃+DMSO-d₆): δ11.35 (s, 1H), 11.05 (s, 1H), 7.75 (s, 1H), 7.52 (s, 1H) and 3.85 (s, 3H).

Methyl 7-bromo-2-oxo-2,3-dihydro-1H-benzo[d]imidazole-5-carboxylate (238 mg, 0.878 mmol) and 2 N aqueous sodium hydroxide (1.50 mL, 3.0 mmol) were combined in methanol (5 mL) and heated to 50° C. for 90 minutes. The reaction solution was concentrated to remove the methanol. To the reaction residue was added ethyl acetate (5 mL). The resultant solution was acidified with 1N aqueous hydrochloric acid (1.5 mL) to give a final pH of 4. A precipitate formed which was filtered and dried under vacuum to give the title compound (226 mg, 100%) as a solid. ¹H NMR (400 MHz, CD₃OD) ppm 7.89 (d, J=1.37 Hz, 1H) 7.65 (d, J=1.37 Hz, 1H).

Acid Preparation 16: 2-(3-methyl-1,2,4-oxadiazol-5-yl)isonicotinic Acid

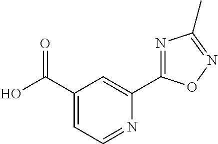

A mixture of acetonitrile (2 mol), hydroxylamine hydrochloride (2 mol) and sodium methoxide (2 mol) was stirred at room temperature for 3 days, then filtered and the filtrate concentrated below 20° C. to give (Z)-N′-hydroxyacetimidamide (150 g) as a white solid which was directly used in the next step.

A mixture of methanol (800 mL), potassium hydroxide (44 g, 0.95 mol) and dimethyl pyridine-2,4-dicarboxylate (ChemPacific) (156 g, 0.79 mol) was refluxed for 0.5 hours and then evaporated in vacuo to afford 4-(methoxycarbonyl)picolinic acid (144 g) as a yellow solid.

To 4-(methoxycarbonyl)picolinic acid (150 g, 1.62 mol) in dichloromethane (500 mL) was added oxalyl chloride (400 mL) keeping the temperature at 25-30° C. for 3 days. The reaction was evaporated in vacuo to afford methyl 2-(chlorocarbonyl)isonicotinate as yellow oil.

To a solution of methyl 2-(chlorocarbonyl)isonicotinate in dichloromethane (500 mL) was added (Z)-N′-hydroxyacetimidamide and triethylamine, keeping the temperature at 25-30° C. for 1 day. The reaction was concentrated in vacuo to afford (Z)-methyl 2-((1-aminoethylideneaminooxy)carbonyl)isonicotinate as a yellow solid.

A solution of (Z)-methyl 2-((1-aminoethylideneaminooxy)carbonyl)isonicotinate in toluene (1 L) was heated at reflux overnight. The obtained mixture was evaporated and purified by silica-gel column chromatography to afford methyl 2-(3-methyl-1,2,4-oxadiazol-5-yl)isonicotinate as a white solid.

A mixture of lithium hydroxide (15 g, 0.35 mol), ethanol (500 mL) and methyl 2-(3-methyl-1,2,4-oxadiazol-5-yl)isonicotinate (52 g, 0.23 mol) were stirred at room temperature for 5 hours, then mixture was concentrated in vacuo. Water was added then extracted with ethyl acetate. The water layer was brought to pH 1.5 with aqueous 1N hydrochloric acid and extracted with ethyl acetate. The organic layer was concentrated in vacuo to afford the title compound as a white solid (42 g). ¹H NMR (300 MHz, DMSO-d₆) ppm 14.08 (br s, 1H) 9.00-8.98 (m, 1H) 8.50 (s, 1H) 8.09-8.07 (m, 1H), 2.46 (s, 3H).

Preparation I-1A-0: tert-butyl 9-oxo-3-azaspiro[5.5]undec-7-ene-3-carboxylate

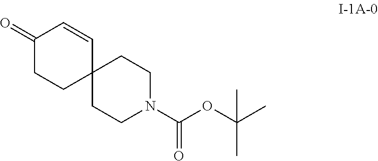

Methyl vinyl ketone (146 mL) was added to a solution of tert-butyl 4-formylpiperidine-1-carboxylate (375 g) in tetrahydrofuran (18 L). The reaction mixture was cooled to −5° C. and a solution of potassium hydroxide in ethanol (3N, 0.243 L) was added dropwise over 10 minutes. The reaction mixture was allowed to warm to room temperature and stirred for 16 hours. Cyclohexane (10 L) was added and the solution was washed with saturated sodium chloride (3×10 L). The organic layer was concentrated to an oil. This oil was dissolved in 2L of 80:20 cyclohexane/ethyl acetate and filtered through Celite® to remove insoluble material. The filtrate was purified via flash column chromatography (70:30 hexane/ethyl acetate) to afford the product as an oil. The oil was triturated in hexanes to afford the desired product as a white solid (131 g, 28%).

Preparation I-1A-1: benzyl 9-oxo-3-azaspiro[5.5]undec-7-ene-3-carboxylate

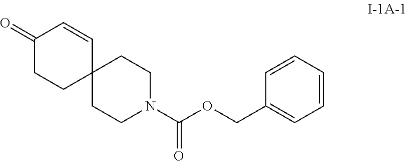

To a benzene (700 mL) solution of benzyl 4-formylpiperidine-1-carboxylate (90.0 g, 363.9 mmol) stirring in a 2 L 3-neck flask fitted with a Dean-Stark trap was added p-toluenesulfonic acid (6.92 g, 36.4 mmol). The reaction was heated to 70° C., 3-buten-2-one (61.8 mL, 753 mmol) was added and mixture was heated at reflux for 24 hours collecting expelled water in the trap. The reaction was cooled to room temperature and washed with 500 mL saturated aqueous sodium bicarbonate. The organic phase was dried over sodium sulfate, filtered and concentrated. The resultant dark brown oil was taken up in 200 mL dichloromethane and filtered through a silica pad (600 mL silica), eluting with 2 L heptane followed by 3 L 50% ethyl acetate/heptane and then 3 L ethyl acetate, collecting by 1 L fractions. Fractions containing clean product were combined and concentrated to yield 68.1 g of the title compound as a thick brown oil. The fractions containing impure product were combined and concentrated and purified by flash chromatography (10-80% ethyl acetate/heptane, 340 g silica gel) to yield an additional 23.6 g of the title compound as a thick brown oil. Combined yield of 91.7 g, (94.1%) was realized. ¹H NMR (400 MHz, CDCl₃) δ ppm 7.27-7.43 (m, 5H), 6.79 (d, J=10.3 Hz, 1H), 5.95 (d, J=10.3 Hz, 1H), 5.13 (s, 2H), 3.56-3.71 (m, 2H), 3.39-3.55 (m, 2H), 2.38-2.50 (m, 2H), 1.96 (t, J=6.7 Hz, 2H), 1.70-1.52 (m, 4H).

Preparation I-1A-1a: (E)-benzyl 9-(2-tert-butylhydrazono)-3-azaspiro[5.5]undec-7-ene-3-carboxylate Hydrochloride Salt

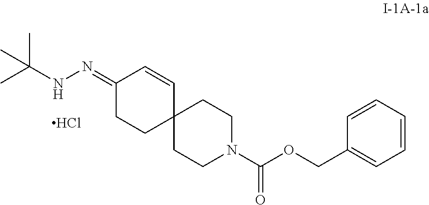

Benzyl 9-oxo-3-azaspiro[5.5]undec-7-ene-3-carboxylate, Preparation I-1A-1 (4.89 g, 16.3 mmol) was dissolved in 60 mL ethanol and tert-butylhydrazine hydrochloride (2.44 g, 19.6 mmol) was added. The mixture was heated at reflux for 4 hours and then stirred at 60° C. for 48 hours. The reaction was cooled to room temperature and concentrated under reduced pressure to yield a tan oil which solidified on standing to yield 6.60 g (99%) of the title compound as a tan solid. ¹H NMR (400 MHz, CDCl₃) ppm 7.26-7.42 (m, 5H), 6.46 (d, J=10.0 Hz, 1H), 6.26 (br. s., 1H), 5.08-5.16 (m, 2H), 3.43-3.58 (m, 4H), 3.19 (s, 2H), 1.78 (s, 2H), 1.44-1.63 (m, 4H), 1.17-1.30 (m, 9H); +ESI MS (M+H)=370.3.

Preparation I-1A-1b: benzyl 2-tert-butyl-2,4-dihydrospiro[indazole-5,4′-piperidine]-1′-carboxylate

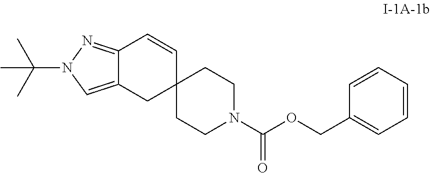

Preparation I-1A-1a (8.00 g, 19.7 mmol) was dissolved in 100 mL dichloromethane and treated with sodium bicarbonate (1.7 g, 19.7 mmol). Stirred 30 minutes and filtered off the sodium chloride formed and concentrated under reduced pressure to yield (E)-benzyl 9-(2-tert-butylhydrazono)-3-azaspiro[5.5]undec-7-ene-3-carboxylate. A 250 mL round bottom flask was charged with 80 mL dimethyl formamide and cooled to 0° C. Phosphorous oxychloride (5.51 mL, 59.1 mmol) was added, dropwise, over 2 minutes and stirred 30 minutes at 0° C. To this solution was added the (E)-benzyl 9-(2-tert-butylhydrazono)-3-azaspiro[5.5]undec-7-ene-3-carboxylate in 15 mL DMF and the reaction was heated at 80° C. for 18 hours. The reaction was cooled to room temperature and concentrated under reduced pressure. The resultant oil was dissolved in 500 mL ethyl acetate and washed with 2×150 mL brine. The aqueous layer was extracted with an additional 100 mL ethyl acetate. The combined organic layers were dried over sodium sulfate, filtered and concentrated. The resultant oil was purified by flash chromatography (10-80% ethyl acetate/heptane gradient, 100 g silica) to yield 4.89 g (65%) of the title compound as a pale yellow oil. ¹H NMR (400 MHz, CDCl₃) ppm 7.25-7.36 (m, 5H), 7.18 (s, 1H), 6.57 (d, J=10.0 Hz, 1H), 5.86 (d, J=10.0 Hz, 1H), 5.12 (s, 2H), 3.51-3.69 (m, 2H), 3.36-3.53 (m, 2H), 2.58 (s, 2H), 1.59-1.74 (m, 2H), 1.52-1.58 (m, 9H), 1.41-1.53 (m, 2H); +ESI MS (M+H)=380.0.

Preparation I-1A-1c: benzyl 6-bromo-2-tert-butyl-7-methoxy-2,4,6,7-tetrahydrospiro[indazole-5,4′-piperidine]-1′-carboxylate

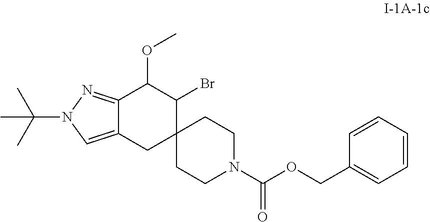

Preparation I-1A-1 b (560 mg, 1.48 mmol) was dissolved in 25 mL of a 20 methanol/tetrahydrofuran mixture. N-bromosuccinimide (315 mg, 1.77 mmol) was added and the mixture was stirred for 30 minutes. The mixture was concentrated under reduced pressure. The resultant oil was partitioned between 50 mL ethyl acetate and 50 mL water. The organic phase was dried over sodium sulfate, filtered and concentrated. The resultant oil was purified by flash chromatography (10-80% ethyl acetate/heptane gradient, 25 g silica) to yield 538 mg (73%) of the title compound as a colorless oil. ¹H NMR (400 MHz, CDCl₃) ppm 7.27-7.43 (m, 6H), 5.12 (s, 2H), 4.74 (d, J=2.7 Hz, 1H), 4.41 (d, J=2.5 Hz, 1H), 3.60-3.84 (m, 2H), 3.54-3.61 (m, 3H), 3.14-3.39 (m, 2H), 2.59 (s, 2H), 1.86 (br. s., 1H), 1.69 (br. s., 3H), 1.51-1.60 (m, 9H).

Preparation 1-1A-1d: benzyl 2-tert-butyl-7-oxo-2,4,6,7-tetrahydrospiro[indazole-5,4′-piperidine]-1′-carboxylate

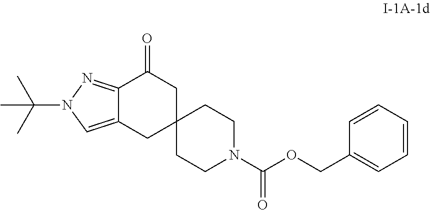

Preparation I-1A-1c (150 mg, 0.31 mmol) was dissolved in 5 mL tetrahydrofuran and treated with potassium tert-butoxide (0.61 mL, 0.61 mmol, 1 M tetrahydrofuran) and stirred 30 minutes. Aqueous 2 N HCl (5 mL) was added and the mixture stirred 15 min at room temperature. Diluted with 50 mL water and extracted with 50 mL ethyl acetate. The organic layer was dried over sodium sulfate, filtered and concentrated. The residue was purified by flash chromatography (10 g silica, 10-80% ethyl acetate/heptane gradient) to yield 86 mg (71%) of the title compound as a clear oil. ¹H NMR (400 MHz, CDCl₃) ppm 7.38 (s, 1H), 7.27-7.35 (m, 5H), 5.11 (s, 2H), 3.48 (t, J=5.8 Hz, 4H), 2.71 (s, 2H), 2.57 (s, 2H), 1.57-1.66 (m, 9H), 1.47-1.59 (m, 4H); +ESI MS (M+H)=396.5

Preparation 1-1A-1e: 2-tert-butyl-4,6-dihydrospiro[indazole-5,4′-piperidin]-7(2H)-one Hydrochloride Salt

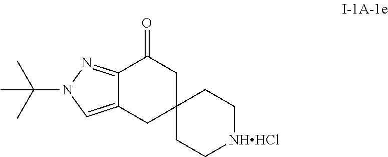

Preparation I-1A-1d (441 mg, 1.12 mmol) was dissolved in 15 mL methanol and treated with ammonium formate (217 mg, 3.34 mmol) and palladium on carbon (50 mg, 10% Pd, 50% H₂O). The reaction was stirred 2 hours at room temperature and the catalyst then removed by filtration. The filtrate was concentrated under reduced pressure. The resultant colorless solid was taken up in 20 mL ethyl acetate and treated with 1 mL 0.5 M HCl in diethyl ether. The mixture was stirred for 30 min and concentrated under reduced pressure. The resultant colorless solid was triturated with 20 mL heptane to yield 265 mg (80%) of the title compound as a colorless solid. ¹H NMR (400 MHz, CD₃OD) ppm 7.74 (s, 1H) 3.20 (t, J=6.1 Hz, 4 H) 2.88 (s, 2H) 2.64 (s, 2H) 1.67-1.91 (m, 4H) 1.55-1.63 (m, 9H). +ESI MS (M+H)=262.1.

Preparation of benzyl 2-tert-butyl-6-methyl-7-oxo-2,4,6,7-tetrahydrospiro[indazole-5,4′-piperidine]-1′-carboxylate (I-1A-3a) and benzyl 2-tert-butyl-6,6-dimethyl-7-oxo-2,4,6,7-tetrahydrospiro[indazole-5,4′-piperidine]-1′-carboxylate (I-1A-4a):

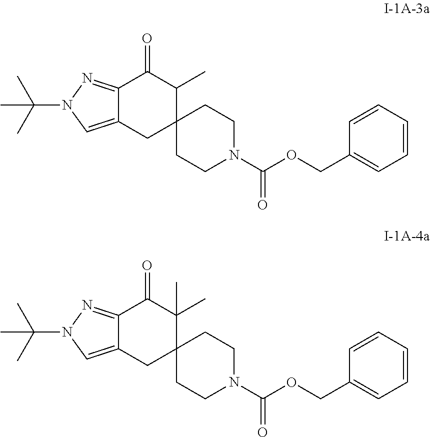

Preparation I-1A-1d (261 mg, 0.66 mmol) was dissolved in 5 mL tetrahydrofuran and cooled to −78° C. A solution of lithium bis(trimethylsilyl)amide (2.64 mL, 2.64 mmol, 1 M THF) was added and stirred 30 minutes at −78° C. and then 10 minutes at 0° C. The mixture was cooled to −78° C. and treated with methyl iodide (0.12 mL, 1.98 mmol). Mixture was stirred 1h while warming to room temperature. The reaction was quenched with 5 mL saturated aqueous ammonium chloride. The mixture was diluted with 50 mL water and extracted with 100 mL ethyl acetate. The organic phase was dried over sodium sulfate, filtered and concentrated. The resultant oil was purified by flash chromatography (10-80% gradient ethyl acetate/heptane, 25 g silica) to yield 110 mg (41%) of racemic I-1A-3a as a colorless oil. ¹H NMR (400 MHz, CDCl₃) δ ppm 7.26-7.41 (m, 6H), 5.11 (s, 2 H), 3.60-3.76 (m, 2H), 3.18-3.41 (m, 2H), 2.62-2.78 (m, 2H), 2.51 (q, J=7.2 Hz, 1H), 1.59-1.65 (m, 9H), 1.47-1.59 (m, 4H), 1.15 (d, J=7.2 Hz, 3H). +ESI MS (M+H)=410.2.

Addition material consisting of a 1:1 mixture of Preparation I-1A-1d and Preparation I-1A-4a (60 mg) was also isolated. This mixture was re-subjected to the above reaction conditions to yield 40 mg (14%) of Preparation I-1A-4a as a clear oil. 1H NMR (400 MHz, CDCl₃) ppm 7.26-7.42 (m, 6H), 5.11 (s, 2H), 3.99 (br. s., 2H), 2.93 (br. s., 3H), 1.36-1.77 (m, 13H), 1.20-1.32 (m, 2H), 1.15 (s, 6H). +ESI MS (M+H)=424.4.

Preparation I-1A-3b: 2-tert-butyl-6-methyl-4,6-dihydrospiro[indazole-5,4′-piperidin]-7(2H)-one Hydrochloride Salt

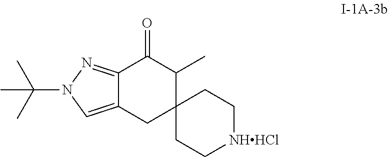

Preparation I-1A-3b was prepared analogous to the synthesis of Preparation I-1A-1d. ¹H NMR (400 MHz, CD₃OD) ppm 7.73 (s, 1H), 3.14-3.27 (m, 4H), 2.89 (s, 2H), 2.51 (q, J=7.2 Hz, 1H), 1.67-1.89 (m, 4H), 1.55-1.62 (m, 9H), 1.17 (d, J=7.4 Hz, 3H); −ESI MS (M+H)=276.5.

Preparation I-1A-4b: 2-tert-butyl-6,6-dimethyl-4,6-dihydrospiro[indazole-5,4′-piperidin]-7(2H)-one Hydrochloride Salt

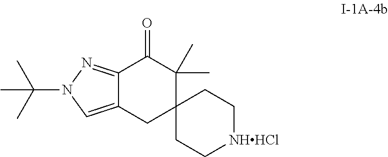

Preparation I-1A-4b was prepared analogous to the synthesis of Preparation I-1A-1d. ¹H NMR (400 MHz, CD₃OD) ppm 7.71 (s, 1H) 3.08-3.26 (m, 4H) 1.81-2.04 (m, 4H) 1.59 (s, 9H) 1.21-1.33 (m, 2H) 1.10-1.22 (m, 6H).

### EXAMPLE 1

Preparation of 2-tert-butyl-1-(1H-indazole-5-carbonyl)-4,6-dihydrospiro[indazole-5,4′-piperidin]-7(2H)-one (1A-1):

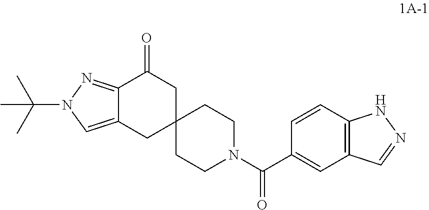

Preparation I-1A-1e (265 mg, 0.89 mmol) and 1H-indazole-5-carbonyl chloride hydrochloride (212 mg, 0.98 mmol) were suspended in 10 mL dichloromethane and N,N-diisopropylethyl amine (0.62 mL, 3.56 mmol) was added dropwise. The mixture was stirred at ambient temperature for 18 hours. The mixture was diluted with 150 mL dichloromethane and washed with brine. The organic phases was dried over sodium sulfate, filtered and concentrated. The resultant oil was taken up in 25 mL methanol and treated with 300 mg potassium carbonate. The mixture was stirred 30 minutes at room temperature. The methanol was removed under reduced pressure and the resultant oil was partitioned between 100 mL ethyl acetate and water. The organic phase was dried over sodium sulfate, filtered and concentrated and then purified by flash chromatography eluting with ethyl acetate to yield 126 mg (35%) of the title compound as a colorless powder. ¹H NMR (400 MHz, CD₃OD) ppm 8.12 (s, 1H), 7.88 (s, 1H), 7.71 (s, 1H), 7.59 (d, J=8.8 Hz, 1H), 7.43 (dd, J=8.6, 1.6 Hz, 1H), 3.44-3.92 (m, 4H), 2.87 (s, 2H), 2.64 (s, 2H), 1.47-1.78 (m, 13H). +ESI MS (M+H)=406.3.

The compounds listed in Table 1 below were prepared using procedures analogous to those described above for the synthesis of Compound 1A-1 using the appropriate starting materials which are available commercially, prepared using preparations well-known to those skilled in the art, or prepared in a manner analogous to routes described above for other intermediates. The compounds listed below were isolated initially as the free base and may be converted to their corresponding hydrochloride salt for testing.

TABLE 1

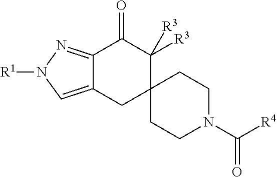

| Ex. | R¹ | R³ | R³ | —C(O)R⁴ | Analytical Data |
|---|---|---|---|---|---|
| 1A-2 | C(CH₃)₃ | H | H | 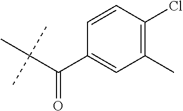 | |
| 1A-3 | C(CH₃)₃ | Me | H | 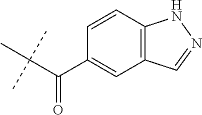 | ¹H NMR (400 MHz, CD₃OD) ppm 8.12 (s, 1 H) 7.82- 7.97 (m, 1 H) 7.70 (s, 1 H) 7.59 (dd, J = 8.6, 1.0 Hz, 1 H) 7.44 (td, J = 8.5, 1.4 Hz, 1 H) 3.34-3.51 (m, 2 H) 3.00- 3.17 (m, 2 H) 2.76-2.99 (m, 2 H) 2.52 (q, J = 7.3 Hz, 1 H) 1.58 (s, 13 H) 1.16 (d, J = 7.0 Hz, 6 H); +ESI MS (M + H) = 420.5 |
| 1A-4 | C(CH₃)₃ | Me | Me | 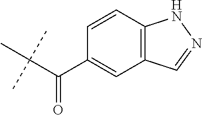 | ¹H NMR (400 MHz, CD₃OD) ppm 8.12 (s, 1 H) 7.84- 7.90 (m, 1 H) 7.70 (s, 1 H) 7.60 (d, J = 8.8 Hz, 1 H) 7.42 (dd, J = 8.8, 1.4 Hz, 1 H) 2.90- 3.27 (m, 4 H) 1.74 (br. s., 4 H) 1.59 (s, 11 H) 1.08- 1.22 (m, 6 H); +ESI MS (M + H) = = 434.5 |

### EXAMPLE 2

Preparation of (ent1)-2-tert-butyl-1′-(1H-indazole-5-carbonyl)-6-methyl-4,6-dihydrospiro[indazole-5,4′-piperidin]-7(2H)-one one (2A-1) and (ent2)-2-tert-butyl-1′-(1H-indazole-5-carbonyl)-6-methyl-4,6-dihydrospiro[indazole-5,4′-piperidin]-7(2H)-one (2A-2):

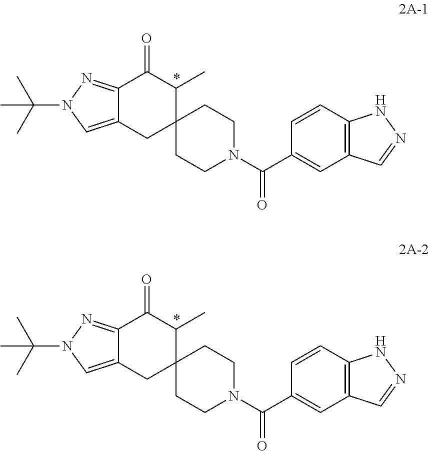

Racemic Example 1A-3 was separated to give the corresponding two enantiomers using chiral HPLC: [Chiralpakl OJ-H (10×250); mobile phase: 85:15 (CO₂/Methanol); flow rate=10 mL/min]. Ent1: retention time=4.29 min; Ent2: retention time=5.88 min.

### PHARMACOLOGICAL DATA

### Biological Protocols

The utility of the compound of present invention, in the treatment of diseases (such as are detailed herein) in animals, particularly mammals (e.g., humans) may be demonstrated by the activity thereof in conventional assays known to one of ordinary skill in the art, including the in vitro and in vivo assays described below. Such assays also provide a means whereby the activities of the compound of the present invention can be compared with the activities of other known compounds.

### Direct Inhibition of the Activities of ACC1 and ACC2

The ACC inhibitory activity of the compound of the present invention was demonstrated by methods based on standard procedures. For example, direct inhibition of ACC activity, for the compound of Formula (I) was determined using preparations of recombinant human ACC1 (rhACC1) and recombinant human ACC2 (rhACC2). Representative sequences of the recombinant human ACC1 and ACC2 that can be used in the assay are provided in FIG. 1 (SEQ ID NO. 1) and FIG. 2 (SEQ. ID NO. 2), respectively.

[1] Preparation of rhACC1. Two liters of SF9 cells, infected with recombinant baculovirus containing full length human ACC1 cDNA, were suspended in ice-cold lysis buffer (25 mM Tris, pH 7.5; 150 mM NaCl; 10% glycerol; 5 mM imidazole (EMD Bioscience; Gibbstown, NJ); 2mM TCEP (BioVectra; Charlottetown, Canada); Benzonase nuclease (10000 U/100 g cell paste; Novagen; Madison, Wisc.); EDTA-free protease inhibitor cocktail (1 tab/50 mL; Roche Diagnostics; Mannheim, Germany). Cells were lysed by 3 cycles of freeze-thaw and centrifuged at 40,000×g for 40 minutes (4° C.). Supernatant was directly loaded onto a HisTrap FF crude column (GE Healthcare; Piscataway, N.J.) and eluted with an imidazole gradient up to 0.5 M over 20 column volumes (CV). ACC1-containing fractions were pooled and diluted 1:5 with 25 mM Tris, pH 7.5, 2mM TCEP, 10% glycerol and direct loaded onto a CaptoQ (GE Healthcare) column and eluted with an NaCl gradient up to 1 M over 20 CV's. Phosphate groups were removed from purified ACC1 by incubation with lambda phosphatase (100U/10 pM target protein; New England Biolabs; Beverly, Mass.) for 14 hours at 4° C.; okadaic acid was added (1 μM final concentration; Roche Diagnostics) to inhibit the phosphatase. Purified ACC1 was exchanged into 25 mM Tris, pH 7.5, 2 mM TCEP, 10% glycerol, 0.5 M NaCl by 6 hour dialysis at 4° C. Aliquots were prepared and frozen at −80° C.

[2] Measurement of rhACC1 inhibition. hACC1 was assayed in a Costar #3676 (Costar, Cambridge, Mass.) 384-well plate using the Transcreener ADP detection FP assay kit (Bellbrook Labs, Madison, Wisc.) using the manufacturer's recommended conditions for a 50 μM ATP reaction. The final conditions for the assay were 50 mM HEPES, pH 7.2, 10 mM MgCl₂, 7.5 mM tripotassium citrate, 2 mM DTT, 0.1 mg/mL BSA, 30 μM acetyl-CoA, 50 μM ATP, and 10 mM KHCO₃. Typically, a 10 μl reaction was run for 120 min at 25° C., and 10 μl of Transcreener stop and detect buffer was added and the combination incubated at room temp for an additional 1 hour. The data was acquired on a Envision Fluorescence reader (Perkinelmer) using a 620 excitation Cy5 FP general dual mirror, 620 excitation Cy5 FP filter, 688 emission (S) and a 688 (P) emission filter.

[3] Preparation of rhACC2.Human ACC2 inhibition was measured using purified recombinant human ACC2 (hrACC2). Briefly, a full length Cytomax clone of ACC2 was purchased from Cambridge Bioscience Limited and was sequenced and subcloned into PCDNA5 FRT TO-TOPO (Invitrogen, Carlsbad, Calif.). The ACC2 was expressed in CHO cells by tetracycline induction and harvested in 5 liters of DMEM/F12 with glutamine, biotin, hygromycin and blasticidin with1 g/mL tetracycline (Invitrogen, Carlsbad, Calif.). The conditioned medium containing ACC2 was then applied to a Softlink Soft Release Avidin column (Promega, Madison, Wisc.) and eluted with 5 mM biotin. 4 mgs of ACC2 were eluted at a concentration of 0.05 mg/mL (determined by A280) with an estimated purity of 95% (determined by A280). The purified ACC2 was dialyzed in 50 mM Tris, 200 mM NaCl, 4 mM DTT, 2 mM EDTA, and 5% glycerol. The pooled protein was frozen and stored at −80° C., with no loss of activity upon thawing. For measurement of ACC2 activity and assessment of ACC2 inhibition, test compounds were dissolved in DMSO and added to the rhACC2 enzyme as a 5× stock with a final DMSO concentration of 1%.

[4] Measurement of human ACC2 inhibition. hACC2 was assayed in a Costar #3676 (Costar, Cambridge, Mass.) 384-well plate using the Transcreener ADP detection FP assay kit (Bellbrook Labs, Madison, Wisc.) using the manufacturer's recommended conditions for a 50 uM ATP reaction. The final conditions for the assay were 50 mM HEPES, pH 7.2, 5 mM MgCl₂, 5 mM tripotassium citrate, 2 mM DTT, 0.1 mg/mL BSA, 30 μM acetyl-CoA, 50 μM ATP, and 8 mM KHCO₃. Typically, a 10 μl reaction was run for 50 min at 25° C., and 10 μl of Transcreener stop and detect buffer was added and the combination incubated at room temp for an additional 1 hour. The data was acquired on an Envision Fluorescence reader (Perkinelmer) using a 620 excitation Cy5 FP general dual mirror, 620 excitation Cy5 FP filter, 688 emission (S) and a 688 (P) emission filter.

The results using the recombinant hACC1 and recombinant hACC2 Transcreener assays described above are summarized in the table below for the Compounds of Formula (I) exemplified in the Examples above.

Acute in vivo Assessment of ACC Inhibition in Experimental Animals

The ACC inhibitory activity of the compound of the present invention can be confirmed in vivo by evaluation of their ability to reduce malonyl-CoA levels in liver and muscle tissue from treated animals.

Measurement of malonyl-CoA production inhibition in experimental animals. In this method, male Sprague-Dawley Rats, maintained on standard chow and water ad libitum (225-275 g), were randomized prior to the study. Animals were either fed, or fasted for 18 hours prior to the beginning of the experiment. Two hours into the light cycle the animals were orally dosed with a volume of 5 mL/kg, (0.5% methyl cellulose; vehicle) or with the appropriate compound (prepared in vehicle). Fed vehicle controls were included to determine baseline tissue malonyl-CoA levels while fasted animals were included to determine the effect fasting had on malonyl-CoA levels. One hour after compound administration the animals were asphyxiated with CO₂ and the tissues were removed. Specifically, blood was collected by cardiac puncture and placed into BD Microtainer tubes containing EDTA (BD Biosciences, N.J.), mixed, and placed on ice. Plasma was used to determine drug exposure. Liver and quadriceps were removed, immediately freeze-clamped, wrapped in foil and stored in liquid nitrogen.

Tissues were pulverized under liquid N₂ to ensure uniformity in sampling. Malonyl-CoA was extracted from the tissue (150-200 mg) with 5 volumes 10% tricarboxylic acid in Lysing Matrix A (MP Biomedicals, PN 6910) in a FastPrep FP120 (Thermo Scientific, speed=5.5; for 45 seconds). The supernatant containing malonyl-CoA was removed from the cell debris after centrifugation at 15000×g for 30 minutes (Eppendorf Centrifuge 5402). Samples were stably frozen at −80 C until analysis is completed.

Analysis of malonyl CoA levels in liver and muscle tissue can be evaluated using the following methodology.

The method utilizes the following materials: Malonyl-CoA tetralithium salt and malonyl-¹³C₃-CoA trilithium salt which were purchased from Isotec (Miamisburg, Ohio, USA), sodium perchlorate (Sigma, cat no. 410241), trichloroacetic acid (ACROS, cat no. 42145), phosphoric acid (J. T. Baker, cat no. 0260-01), ammonium formate (Fluka, cat no. 17843), methanol (HPLC grade, J. T. Baker, cat no. 9093-33), and water (HPLC grade, J. T. Baker, 4218-03) were used to make the necessary mobile phases. Strata-X on-line solid phase extraction columns, 25 μm, 20 mm×2.0 mm I.D (cat no. 00M-S033-B0-CB) were obtained from Phenomenex (Torrance, Calif., USA). SunFire C18 reversed-phase columns, 3.5 μm, 100 mm×3.0 mm I.D. (cat no.186002543) were purchased from Waters Corporation (Milford, Mass., USA).

This method may be performed utilizing the following equipment. Two-dimensional chromatography using an Agilent 1100 binary pump, an Agilent 1100 quaternary pump and two Valco Cheminert 6-port two position valves. Samples were introduced via a LEAP HTC PAL auto sampler with Peltier cooled stack maintained at 10° C. and a 20 μL sampling loop. The needle wash solutions for the autosampler are 10% trichloroacetic acid in water (w/v) for Wash 1 and 90:10 methanol:water for Wash 2. The analytical column (Sunfire) was maintained at 35° C. using a MicroTech Scientific Micro-LC Column Oven. The eluent was analyzed on an ABI Sciex API3000 triple quadrupole mass spectrometer with Turbo Ion Spray.

Two-dimensional chromatography was performed in parallel using distinct gradient elution conditions for on-line solid phase extraction and reversed-phase chromatography. The general design of the method was such that the first dimension was utilized for sample clean-up and capture of the analyte of interest followed by a brief coupling of both dimensions for elution from the first dimension onto the second dimension. The dimensions were subsequently uncoupled allowing for gradient elution of the analyte from the second dimension for quantification while simultaneously preparing the first dimension for the next sample in the sequence. When both dimensions were briefly coupled together, the flow of the mobile phase in the first dimension was reversed for analyte elution on to the second dimension, allowing for optimal peak width, peak shape, and elution time.

The first dimension of the HPLC system utilized the Phenomenex strata-X on-line solid phase extraction column and the mobile phase consisted of 100 mM sodium perchlorate/0.1% (v/v) phosphoric acid for solvent A and methanol for solvent B.

The second dimension of the HPLC system utilized the Waters SunFire C18 reversed-phase column and the mobile phase consisted of 100 mM ammonium formate for solvent A and methanol for solvent B. The initial condition of the gradient was maintained for 2 minutes and during this time the analyte was transferred to the analytical column. It was important that the initial condition was at a sufficient strength to elute the analyte from the on-line SPE column while retaining it on the analytical. Afterwards, the gradient rose linearly to 74.5% A in 4.5 minutes before a wash and re-equilibration step.

Mass spectrometry when coupled with HPLC can be a highly selective and sensitive method for quantitatively measuring analytes in complex matrices but is still subject to interferences and suppression. By coupling a two dimensional HPLC to the mass spectrometer, these interferences were significantly reduced. Additionally, by utilizing the Multiple Reaction Monitoring (MRM) feature of the triple quadrupole mass spectrometer, the signal-to-noise ratio was significantly improved.

For this assay, the mass spectrometer was operated in positive ion mode with a TurbolonSpray voltage of 2250V. The nebulizing gas was heated to 450° C. The Declustering Potential (DP), Focusing Potential (FP), and Collision Energy (CE) were set to 60, 340, and 42 V, respectively. Quadrupole 1 (Q1) resolution was set to unit resolution with Quadrupole 3 (Q3) set to low. The CAD gas was set to 8. The MRM transitions monitored were for malonyl CoA: 854.1→347.0 m/z (L. Gao et al. (2007) J. Chromatogr. B 853,303-313); and for malonyl-¹³C₃-CoA: 857.1→350.0 m/z with dwell times of 200 ms. The eluent was diverted to the mass spectrometer near the expected elution time for the analyte, otherwise it was diverted to waste to help preserve the source and improve robustness of the instrumentation. The resulting chromatograms were integrated using Analyst software (Applied Biosystems). Tissue concentrations for malonyl CoA were calculated from a standard curve prepared in a 10% solution of trichloroacetic acid in water.

Samples comprising the standard curve for the quantification of malonyl-CoA in tissue extracts were prepared in 10% (w/v) trichloroacetic acid (TCA) and ranged from 0.01 to 1 pmol/μL. Malonyl-¹³C₃-CoA (final concentration of 0.4 pmol/μL) was added to each standard curve component and sample as an internal standard.

Six intra-assay quality controls were prepared; three from a pooled extract prepared from fasted animals and three from a pool made from fed animals. These were run as independent samples spiked with 0, 0.1 or 0.3 pmol/μL ¹²C-malonyl-CoA as well as malonyl-¹³C₃-CoA (0.4 pmol/μL). Each intra-assay quality control contained 85% of aqueous tissue extract with the remaining portion contributed by internal standard (0.4 pmol/μL) and ¹²C-malonyl-CoA. Inter assay controls were included in each run; they consist of one fasted and one fed pooled sample of quadriceps and/or one fasted and one fed pooled sample of liver. All such controls are spiked with malonyl-¹³C₃-CoA (0.4 pmol/μL).

What is claimed is:

1. A compound of Formula (I) 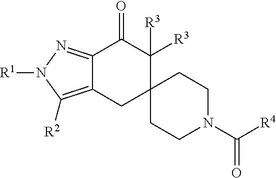
wherein
R¹ is (C₁-C₆)alkyl, (C₃-C₇)cycloalkyl, tetrahydrofuranyl or oxetanyl; wherein said (C₁-C₆)alkyl is optionally substituted with 1 to 2 substituents independently selected from (C₁-C₃)alkoxy, hydroxy, fluoro, phenyl, tetrahydrofuranyl or oxetanyl;
R² is hydrogen, halo, (C₁-C₃)alkyl, or cyano;
R³ are each independently hydrogen or (C₁-C₃)alkyl;
R⁴ is (C₆-C₁₀)aryl, 5 to 12 membered heteroaryl or 8 to 12 membered fused heterocyclicaryl; wherein said (C₆-C₁₀)aryl, 5 to 12 membered heteroaryl or 8 to 12 membered fused heterocyclicaryl are each optionally substituted with one to three substituents independently selected from (C₁-C₃)alkyl, (C₁-C₃)alkoxy, halo, amino, (C₁-C₃)alkylamino, di(C₁-C₃)alkylamino, hydroxy, cyano, amido, phenyl, 5 to 6 membered heteroaryl or 5 to 6 membered heterocyclyl; or a pharmaceutically acceptable salt thereof.

2. The compound of claim 1 wherein R¹ is (C₁-C₆)alkyl, (C₃-C₇)cycloalkyl, or tetrahydrofuranyl; and R² is hydrogen or methyl; or a pharmaceutically acceptable salt thereof.

3. The compound of claim 2 wherein R¹ is ethyl, isopropyl or t-butyl; each R³ is hydrogen; and R⁴ is phenyl, pyrazolyl, imidazolyl, triazolyl, pyridinyl, pyrimidinyl, indolyl, benzopyrazinyl, benzoimidazolyl, benzoimidazolonyl, pyrrolopyridinyl, pyrrolopyrimidinyl, pyrazolopyridinyl, pyrazolopyrimidinyl, indazolyl, indolinonyl, naphthyridinyl, quinolinyl, quinolinonyl, dihydroquinolinonyl, oxo-dihydroquinolinonyl, isoquinolinyl, isoquinolinonyl, dihydroisoquinonyl or oxo-dihydroisoquinonyl, each optionally substituted with one to three substituents independently selected from fluoro, chloro, methyl, methoxy, amino, methylamino, dimethylamino, amido, cyano, phenyl, imidazolyl, pyrazolyl, triazolyl, pyridinyl or morpholinyl; or a pharmaceutically acceptable salt thereof.

4. The compound of claim 3 wherein R¹ is isopropyl or t-butyl; R² is hydrogen; or a pharmaceutically acceptable salt thereof.

5. The compound of claim 4 wherein R⁴ is indazolyl, benzoimidazolyl, 1-oxo-1,2-dihydroisoquinolinyl, 1H-pyrrolo[3,2-b]pyridinyl, 2-oxo-2,3-dihydro-1H-benzo[d]imidazolyl, 1H-pyrazolylphenyl, 1H-pyrazolylpyridinyl, or 1H-imidazolylphenyl; each optionally substituted with one to two methyl, chloro or fluoro; or a pharmaceutically acceptable salt thereof.

6. A compound selected from the group consisting of
2-tert-butyl-1′-(1H-indazole-5-carbonyl)-4,6-dihydrospiro[indazole-5,4′-piperidin]-7(2H)-one;
2-tert-butyl-1′-(4-chloro-3-methyl-phenylcarbonyl)-4,6-dihydrospiro[indazole-5,4′-piperidin]-7(2H)-one;
2-tert-butyl-1′-(1H-indazole-5-carbonyl)-6,6-dimethyl-4,6-dihydrospiro[indazole-5,4′-piperidin]-7(2H)-one;
2-tert-butyl-1′-(1H-indazole-5-carbonyl)-6-methyl-4,6-dihydrospiro[indazole-5,4′-piperidin]-7(2H)-one;
(R)-2-tert-butyl-1′-(1H-indazole-5-carbonyl)-6-methyl-4,6-dihydrospiro[indazole-5,4′-piperidin]-7(2H)-one; and
(S)-2-tert-butyl-1′-(1H-indazole-5-carbonyl)-6-methyl-4,6-dihydrospiro[indazole-5,4′-piperidin]-7(2H)-one;
or a pharmaceutically acceptable salt thereof.

7. The compound of claim 6 selected from
2-tert-butyl-1′-(1H-indazole-5-carbonyl)-4,6-dihydrospiro[indazole-5,4′-piperidin]-7(2H)-one;
(R)-2-tert-butyl-1′-(1H-indazole-5-carbonyl)-6-methyl-4,6-dihydrospiro[indazole-5,4′-piperidin]-7(2H)-one; and
(S)-2-tert-butyl-1′-(1H-indazole-5-carbonyl)-6-methyl-4,6-dihydrospiro[indazole-5,4′-piperidin]-7(2H)-one;
or a pharmaceutically acceptable salt thereof.

8. A pharmaceutical composition comprising a therapeutically effective amount of a compound of claim 1; or a pharmaceutically acceptable salt thereof and a pharmaceutically acceptable excipient, diluent, or carrier.

9. The composition of claim 8 further comprising at least one additional anti-diabetic agent.

10. The composition of claim 9 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

11. A method for treating Type 2 diabetes and diabetes-related disorders in animals comprising the step of administering to an animal in need of such treatment a therapeutically effective amount of a compound of claim 1 or a pharmaceutically acceptable salt thereof.

12. A method for treating nonalcoholic fatty liver disease (NAFLD) or hepatic insulin resistance in animals comprising the step of administering to an animal in need of such treatment a therapeutically effective amount of a compound of claim 1 or a pharmaceutically acceptable salt thereof.

13. A method for treating Type 2 diabetes and diabetes-related disorders in animals comprising the step of administering to an animal in need of such treatment a pharmaceutical composition of claim 8.

14. A method for treating nonalcoholic fatty liver disease (NAFLD) or hepatic insulin resistance in animals comprising the step of administering to an animal in need of such treatment a pharmaceutical composition of claim 8.

15. A method for treating a disease, condition or disorder modulated by the inhibition of acetyl-CoA carboxylase enzyme(s) in animals comprising the step of administering to an animal in need of such treatment two separate pharmaceutical compositions comprising:
(i) a first composition comprising a therapeutic amount of a compound of claim 1 or a pharmaceutically acceptable salt thereof, and a pharmaceutically acceptable excipient, diluent, or carrier; and
(ii) a second composition comprising at least one additional pharmaceutical agent selected from the group consisting of an anti-obesity agent and an anti-diabetic agent; and a pharmaceutically acceptable excipient, diluent, or carrier;
wherein said disease, condition or disorder modulated by the inhibition of acetyl-CoA carboxylase enzyme(s) is selected from the group consisting of obesity, obesity-related disorders, Type 2 diabetes, diabetes-related disorders, nonalcoholic fatty liver disease (NAFLD) and hepatic insulin resistance.
